(19) United States
(12) Patent Application Publication
(10) Pub. No.: US 2009/0246211 A1
(43) Pub. Date: Oct. 1, 2009

(54) Molecular constructs suitable for targeted conjugates

(76) Inventors: John T. Henri, Longmont, CO (US); James D. McChesney, Boulder, CO (US); Rodger Lamb, Westminster, CO (US); Sylesh K. Venkataraman, Boulder, CO (US)

Correspondence Address: MORGAN, LEWIS & BOCKIUS LLP, 1701 MARKET STREET, PHILADELPHIA, PA, 19103-2921, US

(21) Appl. No.: 11/433,071

(22) Filed: May 11, 2006

Related U.S. Application Data
(60) Provisional application No. 60/680,623, filed on May 12, 2005.

(51) Int. Cl. A61K 39/44; C07K 17/06; A61K 47/48; A61K 38/00; A61K 38/10; A61K 38/16; A61P 35/00; A61P 35/02

(52) U.S. Cl. 424/181.1; 530/350; 530/388.1; 530/387.1; 530/327; 530/370; 514/2; 514/12; 514/14

(57) ABSTRACT

The present invention relates generally to effective drug-linker constructs suitable for conjugation with ligands. The present invention also discloses methods of conjugating these constructs with peptides to form the compound of formula I. These methods are readily extended to any hydroxyl, amine or sulfur bearing biologically active molecules.

B—X—L¹—(L²)ₘ—(L³)ₙ—Y—RL I

### CROSS REFERENCE TO RELATED APPLICATIONS

This application claims the benefit of U.S. Provisional Application No. 60/680,623, filed May 12, 2005, which is incorporated herein in its entirety.

### BACKGROUND OF THE INVENTION

A number of anti-cancer drugs are currently in the market for the treatment of various cancers. For example, paclitaxel and docetaxel are two promising anti-cancer drugs used to treat breast and ovarian cancers, and which hold promise for the treatment of various other cancers such as skin, lung, head and neck carcinomas. Other promising chemotherapeutic agents are being developed or tested for treatment of these and other cancers. Compounds such as paclitaxel, docetaxel, and other taxanes, camptothecins, epothilones and quassinoids, as well as other compounds exhibiting efficacy in cancer treatment, are of considerable interest. Of special interest are natural product drugs and their synthetic analogs with demonstrated anticancer activity in vitro and in vivo.

However, many identified anti-cancer compounds present a number of difficulties with their use in chemotherapeutic regimens. One particular problem relates to the aqueous insolubility of many anti-cancer compounds, which creates significant problems in developing suitable pharmaceutical formulations useful for chemotherapy. In an attempt to increase the aqueous solubility of these drugs, they are often formulated with various carrier compounds. However, these carrier compounds often cause various adverse side effects in a patient treated with the formulation. For example, paclitaxel, and camptothecin and their analogs are generally formulated with a mixture of polyethoxylated castor oil (Cremophor) and ethanol. This mixture has been reported to cause side effects in clinical trials, which include neutropenia, mucositis, cardiac and neurological toxicities, hypersensitivity, histamine release and severe allergic reactions.

Another major problem with the use of such chemotherapeutic agents in cancer treatment is the difficulty targeting cancer tissues, without adversely affecting normal, healthy tissues. For example, paclitaxel exerts its antitumor activity by interrupting mitosis and the cell division process, which occurs more frequently in cancer cells, than in normal cells. Nonetheless, a patient undergoing chemotherapy treatment may experience various adverse effects associated with the interruption of mitosis in normal, healthy cells.

Targeted cancer therapies that can selectively kill cancer cells without harming other cells in the body would represent a major improvement in the clinical treatment of cancer. Reports of targeting drugs using antibodies have appeared in the literature since 1958. Targeting drugs by conjugation to antibodies for selective delivery to cancer cells has had limited success due to the large size of antibodies (MW=125-150 kilodaltons) and thus their relative inability to penetrate solid tumors.

An alternative strategy comprises the use of smaller targeting ligands and peptides, which recognize specific receptors unique to or over expressed on tumor cells, as the targeting vector. Such constructs have molecular weights of 2-6 kilodaltons, which allows ready penetration throughout solid tumors.

Accordingly, it would be highly desirable to develop chemical compounds and methods for use in directly targeting cancer cells with chemotherapeutic agents in cancer treatment regimens. This, in turn, could lead to reduction or elimination of toxic side effects from carrier compounds, more efficient delivery of the drug to the targeted site, and reduction in dosage of the administered drug and a resulting decrease in toxicity to healthy cells and in the cost of administering the chemotherapeutic regimen.

One particular approach of interest is the use of anticancer drug moieties that have been conjugated to tumor recognizing molecules. For example, U.S. Pat. No. 6,191,290 to Safavy discusses the formation and use of a taxane moiety conjugated to a receptor ligand peptide capable of binding to tumor cell surface receptors. Safavy in particular indicates that such receptor ligand peptides might be a bombesin/gastrin-releasing peptide (BBN/GRP) receptor-recognizing peptide (BBN[7-13]), a somatostatin receptor-recognizing peptide, an epidermal growth factor receptor-recognizing peptide, a monoclonal antibody or a receptor-recognizing carbohydrate.

One important aspect of synthesizing these drug-ligand conjugates is connecting these two units with a linker or linkers that provide conjugates with the desired characteristics and biological activity, in particular, a conjugate that is more water soluble and has higher biological activity and lower toxicity. In one aspect, the ligand is a receptor ligand. The resulting conjugate should also be sufficiently stable until it reaches the target tissue, and thus maximizing the targeting effect with reduced toxicity to the normal, healthy tissue.

### SUMMARY OF THE INVENTION

The present invention relates generally to effective drug-linker constructs suitable for conjugation with ligands for use in cancer treatment. In one aspect, the ligand is a receptor ligand. In another aspect, the ligand is a peptide, a protein or a targeting peptide with or without internalization. The present invention also discloses methods of conjugating these constructs with peptides. These methods are readily extended to any hydroxyl, amine or sulfur bearing biologically active molecules. Non-exclusive examples of such molecules are taxanes, camptothecins, epothilones, cucurbitacins, quassinoids, anthracyclins, and their analogs and derivatives. Also disclosed herein are methods for the treatment of cancer by the administration of an effective amount of a composition comprising the conjugates in combination with radiotherapy and/or other therapeutic treatments including the co-administration of other chemotherapeutic agents.

The present invention provides new molecular conjugates, including for example, new HN-1-drug conjugates, and Transferrin-drug conjugates, for use in treating cancer in a mammal. In one aspect, the mammal is human. Additionally, the present invention is directed to novel intermediate compounds for use in linking biologically active molecules to carrier molecules such as HN-1, Transferrin or other molecules. The present invention discloses the preparation and use of novel linkers and combination of linkers to prepare various molecular constructs suitable for peptide and protein conjugates. In another aspect, the present invention provides aldehyde ester and amido derivatives, respectively, of hydroxyl-bearing and amine-bearing biologically active molecules, such as cancer therapeutic drugs and analogs and derivatives thereof, as well as precursors thereto, which can be linked to carrier molecules such as human Transferrin protein through the formation of Schiff bases between the aldehyde functionality of the ester or amide linkage with various amino functionalities of the Transferrin molecule or other protein. Optionally, these Schiff's bases can be further reduced to their respective secondary amines to provide the resulting conjugates with increase hydrolytic stability.

Therapeutic benefits may also be realized by the administration of at least one therapeutic conjugate in combination with a second therapy. The therapeutic conjugate of the invention may also be combined with other therapies to provide combined therapeutically effective amounts, as disclosed herein. The treatment methods of the present invention will generally involve the administration of the pharmaceutically effective composition to a subject systemically, such as via intravenous injection. However, any route of administration that allows the therapeutic conjugate of the present invention to localize to the tumor will be effective.

### DETAILED DESCRIPTION OF THE INVENTION

The present invention provides new molecular conjugates, including new HN-1-drug conjugates, Transferrin-drug conjugates and other biologically active molecules, for use in treating cancer in a mammal. In one aspect, the new molecular conjugates provide various compositions of molecular conjugates of hydroxy, amino or sulfur bearing drugs. The conjugates prepared from the present methods display enhanced water solubility and reduction or elimination of toxicity. In a particular aspect, the present invention provides new molecular conjugates of taxane derivatives and analogs, and quassinoid derivatives and analogs that are effective for cancer therapy. In another aspect, the conjugates are also useful as agents in combination therapy with chemotherapeutic agents for cancer therapy.

The present invention also provides aldehyde, ester and amido derivatives, respectively, of hydroxyl-, amine- or sulfur-bearing biologically active molecules, such as cancer therapeutic drugs and analogs and derivatives thereof, as well as precursors thereto, which can be linked to carrier molecules, such as HN-1 or human Transferrin protein either through the formation of thioether linkage between the maleimide functionality of the ester or amide linkage and cysteine of the peptide/protein or through the formation of Schiff bases between the aldehyde functionality of the ester or amide linkage and various amino functionalities of the Transferrin molecule or other protein.

In one aspect, the present invention also provides an efficient protocol for the synthesis of a ligand or carrier molecule conjugates, or other molecular conjugates, of various hydroxyl-bearing, amine-bearing or sulfur-bearing biologically active compounds, and intermediates thereto. A generalized process includes coupling such hydroxyl-bearing, amino-bearing or sulfur-bearing biomolecules with an appropriate acylating agent, such as a carboxylic acid or acid halide, having a double bond, preferably a terminal olefin. Oxidation of the terminal olefin site using, for example, catalytic osmium tetroxide, followed by cleavage of the resulting diol to aldehyde provides a suitable precursor for synthesis of a carrier molecule or ligand or other molecular conjugates. The final step in the synthetic sequence of these adducts is the treatment of the aldehyde with a carrier molecule or a ligand, such as the blood protein Transferrin, or HN-1 peptide, to form biomolecules attached, which are found to have an increased biological activity. These Schiff's base conjugates can be further reduced to their respective secondary amines to form the corresponding conjugates with increased stability toward hydrolysis. The resulting reduced product is particularly advantageous in cases where the compound is designed such that the cleavage of the conjugate does not occur at the imine functional group of the Schiff's base to release the biologically active molecule. In another aspect, the present invention broadly contemplates that the carrier molecule or ligand may include any molecule having at least one accessible amino functionality through which a Schiff base may be formed with the aldehyde functionality of the ester and amido linker compounds of biologically active molecules, as disclosed herein. In yet another aspect, the present invention provides linker chemistry suitable for conjugating the drug-ester-amide constructs linked to ligands via thioether linkages. In one aspect, the process may be achieved by coupling of hydroxyl-bearing, amine-bearing or sulfur-bearing biomolecules first with an appropriate acylating agent, such as a dicarboxylic acid or acid halide, followed by another coupling with a maleimide bearing alcohol. Thioether formation involving the maleimide with the cysteine functionality of the desired peptide/protein such as HN1, BBN provide conjugates of improved biological activity. Upon reaching the target site, these conjugates release the active agent at the desired site resulting in a reduction in side effects caused to normal, healthy tissue.

It should further be understood that, while the focus of the present disclosure is directed to cancer therapy, the present application contemplates the conjugation according to the present invention of various proteins or other carrier molecules with biologically active molecules directed toward other applications.

### Definition:

“Alkyl” means a straight or branched, saturated or unsaturated, aliphatic group comprising carbon atoms in a chain. When a “C₁₋ₙ alkyl” is recited, for example, 1 to n indicate the number of carbon atoms in the chain. Therefore, a C₁₋₄alkyl includes alkyls that have a chain of 1 to 6 carbons atoms, and include such groups as methyl, ethyl, propyl, isopropyl, butyl, sec-butyl, isobutyl, tert-butyl, vinyl, allyl, propargyl, and the like. Each alkyl group may be an unsubstituted hydrocarbon, or may be substituted by one or more substituents as defined herein.

“Amino” is a nitrogen group having two further substituents. Representative amino groups include —NH₂, —NHCH₃, —N(CH₃)₂, —NHCH₂Ph, —NHC(O)CH₃ and the like. In certain aspects, the two substituents together with the nitrogen may form a ring. Depending on the nature of the compounds or intermediates prepared as disclosed herein, the amino group may be substituted with a protecting group.

“Biologically active molecule” includes any molecule that generally affects or is involved in or with one or more biological processes in cells, tissues, vessels, or the like. Such biologically active molecules may comprise drugs, antibodies, antigens, lectins, dyes, stains, tracers or any other such molecule. In particular, hydroxyl-bearing, amino-bearing or sulfur-bearing molecules contemplated for use in the invention include paclitaxel, docetaxel and other taxanes, cholesterol, rhodamine 123, camptothecins, epothilones such as epothilone B, cucurbitacins, quassinoids such as glaucarubolone, brusatol and bruceantin, anthracyclines such as adriamycin, daunorubicin and the like, and their analogs and derivatives, as well as other compounds.

“Electron donating groups” means a group or substituents that have the ability to donate electrons by an inductive effect and/or by a resonance effect. Examples of electron donating groups include —OH, —NH₂, —NHCH₃, alkyl groups, etc.

“Electron withdrawing groups” means a group or substituents that have the ability to withdraw electrons by an inductive effect and/or by a resonance effect. Examples of electron withdrawing groups include —NO₂, chlorine, bromine, iodine, —COOH, —CN, etc.

“Ligand” is any carrier molecule according to the present invention, linked through an amide, amine, ester, ether, thioether or amide Schiff base linkages as disclosed herein. As an example, a “ligand” can be a “receptor ligand” when the ligand is capable of recognizing the targeted receptors. Without being bound by any theory proposed herein, it is proposed that the ligand functions by increasing the concentration of the biologically active molecule in the disease tissue with or without binding to receptors, and it may or may not be internalized into cells.

“Molecular conjugate” or “conjugate” as used herein should be understood to broadly encompass any compound comprising a biologically active molecule linked to a carrier molecule according to the present invention, such as through the amide, amine, ether, ester, amide, Schiff base linkages, and the like as disclosed herein.

“Prodrugs” as used herein means compounds which are rapidly transformed in vivo to the parent compound as disclosed herein, for example, by hydrolysis in blood. A discussion of prodrugs is provided in T. Higuchi and V. Stella, Pro-drugs as Novel Delivery Systems, Vol. 14 of the A.C.S. Symposium Series; and in Edward B. Roche, ed., Bioreversible Carriers in Drug Design, American Pharmaceutical Association and Pergamon Press, 1987, both references of which are incorporated herein by reference. Prodrugs may also be considered to be analogs or derivatives of the compounds.

“Receptor ligand” encompasses any carrier molecule, capable of recognizing targeted receptors with or without internalization; according to the present invention, linked through the amide, amine, ester, ethers, thioethers, amide Schiff base linkages etc . . . as disclosed herein. The present application covers cancer therapy and also contemplates the conjugation, as described herein, of various proteins or other carrier molecules with biologically active molecules that may be directed toward other applications.

“Taxane” and “taxanes” encompasses the class of compounds generically referred to as taxanes and their derivatives and taxoids, including for example, paclitaxel (see generally Merck Index, Monograph No. 7117, 12^{th} Ed. 1996) and docetaxel. Examples of taxoids that can be used to carry out the present invention include, but are not limited to those described in U.S. Pat. Nos. 6,835,746, 6,593,482, 6,500,858, 5,614,645, 6,028,206, 5,411,984 and 5,508,447.

As used herein, compounds or acyclic or cyclic groups that are represented as having dashed bonds, such as the rings having dashed lines below, are intended to represent compounds or rings that may be aromatic rings, partially unsaturated rings, or fully saturated rings. As exemplified in the groups below, for example, in the case of the 6-membered ring, such representation is intended to encompass the substituted cyclohexane, cyclohexene, 1,3-cyclohexadiene, 1,4-cyclohexadiene and their substituted isomers.

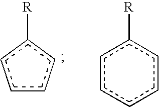

includes

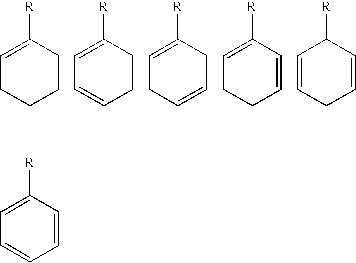

etc . . .

### Aspects of the Invention:

In one embodiment, the present invention provides a compound comprising the formula I:

B—X—L¹—(L²)ₘ—(L³)ₙ—Y—RL I

wherein:

B is a biologically active agent, analog and derivative thereof,

L¹ is a linker comprising at least 2 atoms in the linker chain, wherein the atoms are selected from the group consisting of C, N, O and S;

L² is a linker comprising at least 2 atoms in the linker chain, wherein the atoms are selected from the group consisting of C, N, O and S;

L³ is a linker comprising at least 2 atoms in the linker chain, wherein the atoms are selected from the group consisting of C and N;

X and Y are each independently O, NR¹ or S;

RL is a ligand;

R¹ is hydrogen or a substituted or unsubstituted C₁₋₄alkyl or C₁₋₄alkylCO—;

m is 1, 2, 3, 4 or 5; and n is 0, 1 or 2; or a pharmaceutically acceptable salt or prodrug thereof. In one variation of the above, the biologically active agent is selected from the group consisting of taxanes, camptothecins, epothilones, cucurbitacins, quassinoids, anthracyclines, and their analogs, prodrugs and derivatives. In one variation of the above, the biologically active agent is 2′, 7 or 10-dehydroxyl taxanes, including paclitaxel, docetaxel and compound 70 or 20-dehydroxyl camptothecin or 1, 3 or 7-dehydroxyl epothilones or 11 or 12-dehydroxyl quassinoids and their analogs and derivatives thereof. In each of the embodiments and variations disclosed herein, the “linker” or “linker chain” may comprise of both acyclic groups, cyclic groups, and may be unsaturated, partially saturated, or saturated, and combinations thereof. For example, a C₇ alkyl linker may be a heptylene linker, a benzylene linker or a cyclohexylmethylene linker.

In another embodiment, there is provided a compound comprising the formula II:

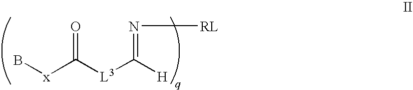

wherein: B is a biologically active agent, analogs and derivatives thereof; RL is a ligand; R¹ is hydrogen or a substituted or unsubstituted C₁₋₄alkyl or C₁₋₄alkylCO—; L³ is selected from the group consisting of (1) a C₁₋₂₀alkyl optionally substituted with one or more phenyl group, the C₁₋₂₀alkyl is optionally interrupted by 1 or 2 heteroatoms selected from the group consisting of O, N or S; (2) a C₃₋₂₀cycloalkyl group optionally substituted with one or more C₁₋₂₀alkyl or phenyl group; and (3) an aromatic group optionally substituted with one or more C₁₋₈alkyl or an electron-withdrawing or electron-donating groups; X is O, NR¹ or S; q is 1-5; or a pharmaceutically acceptable salt or prodrug thereof. In one variation of the above compound, the biologically active agent is a drug useful for cancer therapy.

In another embodiment, there is provided a compound comprising the formula III:

TQ—O—L¹—(L²)ₘ—(L³)ₙ—Y—RL III

wherein TQ is a taxane or quassinoid derivative; L¹ is a linker comprising at least 2 atoms in the linker chain, wherein the atoms are selected from the group consisting of C, N, O and S; L is a linker comprising at least 2 atoms in the linker chain, wherein the atoms are selected from the group consisting of C, N, O and S; L³ is a linker comprising at least 2 atoms in the linker chain, wherein the atoms are selected from the group consisting of C, N, O and S; Y is O, NR¹ or S; RL is a ligand; R¹ is hydrogen or substituted or unsubstituted C₁₋₄alkyl; m is 1, 2, 3, 4 or 5; and n is 0, 1 or 2; or a pharmaceutically acceptable salt or prodrug thereof. In one aspect of the present application, the oxygen atom shown in the above formula III constitute an atom of the taxane or quassinoid derivative. Accordingly, in certain of the above aspects, the taxane or quassinoid derivative represented as “TQ-” may also be referred to as the “des-oxy” taxane derivative or the “des-oxy” quassinoid derivative.

In one variation, the taxane derivative is selected from the group consisting of a paclitaxel, a docetaxel, compound 70 and their derivatives, analogues or prodrugs thereof. In another variation, the taxane derivative is selected from the group consisting of 7-dehydroxyl paclitaxel or docetaxel, 10-dehydroxyl paclitaxel, docetaxel or compound 70, 2′-dehydroxyl paclitaxel, 3′-de-benzamido paclitaxel, and their analogs, prodrugs and derivatives thereof. In the above variation, the taxane derivative is attached to linker L¹. at the 2′-, 7- or 10-hydroxyl position of the taxane moiety. In another variation, the taxane derivative is attached to linker L¹ at the 2′-hydroxyl or at the 7-hydroxyl position of the taxane moiety. In yet another variation, the taxane derivative is attached to linker L¹ at the 2′-hydroxyl position of the taxane moiety. In another variation, the quassinoid derivative is selected from the group consisting of glaucarubolone, bruceantin, brusatol and their derivatives, analogs and prodrugs thereof. In yet another variation of the above, the ligand is selected from the group consisting of an HN-1 peptide, bombesin/gastrin-releasing peptide (BBN/GRP) receptor-recognizing peptide (BBN[7-13]), a somatostatin receptor recognizing peptide, an LHRH receptor recognizing peptide, an epidermal growth factor receptor recognizing peptide, a monoclonal antibody, a receptor recognizing carbohydrate, a receptor ligand peptide and an endogenous peptide that targets the tumors with or without internalization. HN-1 and HN-J are peptides as described by Hong et al., in Cancer Research, 60, 6551, 2000, the reference of which is incorporated herein by reference; according to the present invention these peptides have an additional cysteine group for conjugation with maleimide possessing linkers.

In a particular aspect, the receptor ligand is selected from the group consisting of carrier molecules including peptides, proteins, Transferrin, an antibody, lextins and agents that attaches to the surface of a cell. In one variation of the above compound, L¹ is a substituted or unsubstituted dicarbonyl compound selected from the group consisting of

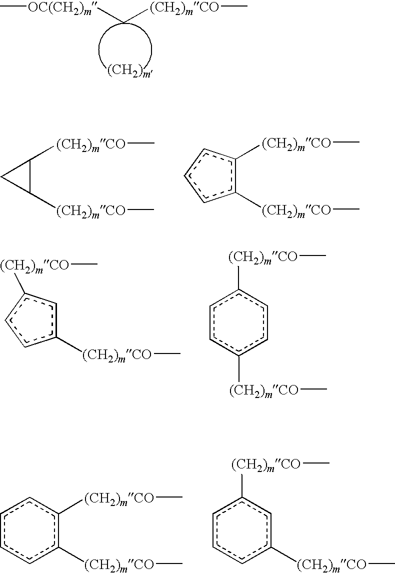

wherein m′ is 2-7; and each m″ is independently 0, 1, 2, 3, 4 or 5.

In another variation of the above, L¹ is selected from the group consisting of substituted or unsubstituted —C(O)(CH₂)_{2—5}C(O)—, —C(O)CH₂CH(CH₃)CH₂C(O)—, —C(O)CH₂C(CH₃)₂CH₂C(O)—, —C(O)CH₂CH(CH₂CH₃)CH₂C(O)—, —C(O)CH₂C(CH₂CH₃)₂CH₂C(O)—, —C(O)(CH₂)₂₋₅CH(CH₃)CH₂C(O)—, —C(O)(CH₂)₂C(CH₃)₂—CH₂C(O)—, —C(O)(CH₂)₂CH(CH₂CH₃)CH₂C(O)— and —C(O)(CH₂)₂C(CH₂CH₃)₂CH₂C(O)—. In another variation, L¹ is selected from the group consisting of substituted or unsubstituted —S(O)(CH₂)_{2—5}S(O)—, —S(O)CH₂CH(CH₃)CH₂S(O)—, —S(O)CH₂C(CH₃)₂CH₂S(O)—, —S(O)CH₂CH(CH₂CH₃)CH₂S(O)—, —S(O)CH₂C(CH₂CH₃)₂CH₂S(O)—, —S(O)(CH₂)₂₋₅CH(CH₃)CH₂S(O)—, —S(O)(CH₂)₂₋₅C(CH₃)₂CH₂S(O)—, —S(O)(CH₂)_{2—5}CH(CH₂CH₃)CH₂S(O)—and —S(O)(CH₂)₂₋₅C(CH₂CH₃)₂CH₂S(O)—. In yet another variation, L¹ is selected from the group consisting of substituted or unsubstituted —C(O)(CH₂)₂₋₅NHC(O)—, —C(O)NH(CH₂)₂₋₅OC(O)—, —C(O)(CH₂)₂₋₅OC(O)—, —C(O)O(CH₂)₂₋₅C(O)—, —C(O)(CH₂)₂₋₅SC(O)— and —C(O)S(CH₂)₂₋₅OC(O)—. In yet another variation, L¹ is selected from the group consisting of substituted or unsubstituted —S(O)(CH₂)₂₋₅NHS(O)—, —S(O)NH(CH₂)₂₋₅OS(O)—, —S(O)(CH₂)₂—SOS(O)— and —S(O)O(CH₂)₂₋₅OS(O)—. In a variation of the above, the substituent is in the position that is alpha to one of the carbonyl, sulfonyl, sulfonamide or sulfone groups. In another variation, the substituent on the dicarbonyl, disulfonyl, sulfone or sulfonamide compound comprises 1, 2 or 3 substituents selected from the group consisting of halo, hydroxy, cyano, aryloxy, silyloxy, C₁₋₄alkyl, C₁₋₄alkoxy, C₁₋₈alkylamino, C₆₋₁₀aryl and C₄₋₁₂heteroaryl.

In another aspect of the above compounds, L² is selected from the group consisting of substituted or unsubstituted —(CH₂)₁₋₅—, —NH(CH₂)₁₋₅—, —NH(CH₂)₂₋₅O—, —O(CH₂CH₂O)₂O—, (CH₂)₂O—, —NH(CH₂)₂(OCH₂CH₂)₃NH—, —NH(CH₂CH₂O)₃(CH₂)₂NHC(O)—, —NH(CH₂CH₂0)₂CH₂CH₂NH—, —NHCH₂CH₂(OCH₂CH₂)₂NHC(O)—, —NH(CH₂)₂O(CH₂)₂NH—, —NH(CH₂)₂O(CH₂)₂NHC(O)—, —O(CH₂CH₂O)₃(CH₂)₂NH—, —O(CH₂CH₂O)₃(CH₂)₂NHC(O)—, —O(CH₂CH₂O)₂(CH₂)₂NH—, —O(CH₂CH₂O)₂(CH₂)₂NHC(O)—, —OCH₂CH₂O(CH₂)₂NH— and —O(CH₂)₂O(CH₂)₂NHC(O)—. In one variation, L is selected from the group consisting of substituted or unsubstituted —OCH(CH₃)C(O)—, —O(CH₂)₃₋₆—, —O(CH₂)₂₋₆NH—, —OCH₂CH₂O—, —O(CH₂)₃₋₆O—, —NH(CH₂)₂₋₆—, —NH(CH₂)₂₋₆NH— and —NH(CH₂)₂₋₆O—. In another variation, L² is —C(O)CH(OR²)CH(OR³)C(O)—, wherein R² and R³ are each independently hydrogen or C₁₋₄alkyl.

In one aspect of the above compound, L³ is a moiety selected from the group consisting of substituted or unsubstituted

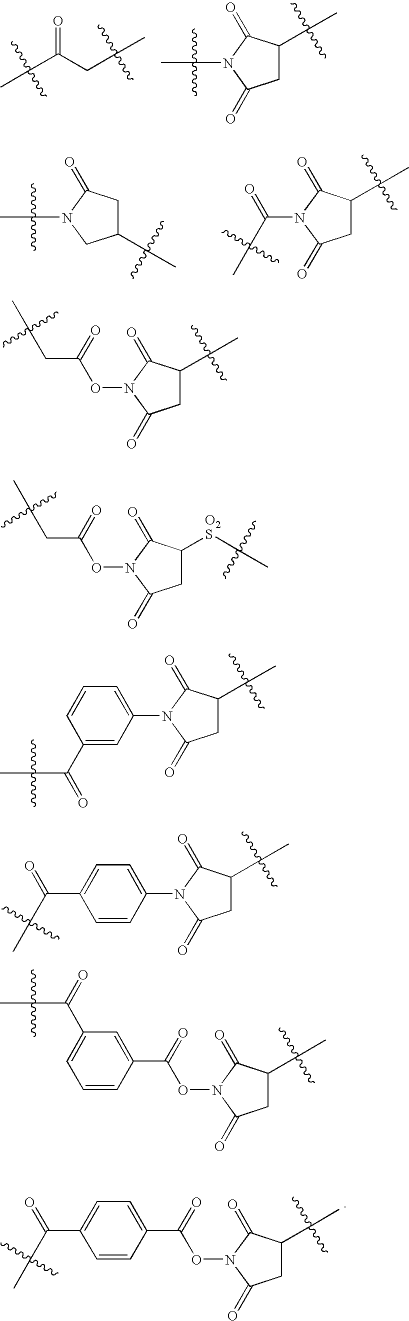

In another variation, L³ is a moiety selected from the group consisting of substituted or unsubstituted

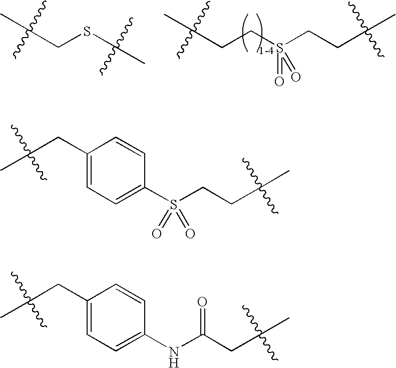

or, wherein L³ is a moiety selected from the group consisting of substituted or unsubstituted

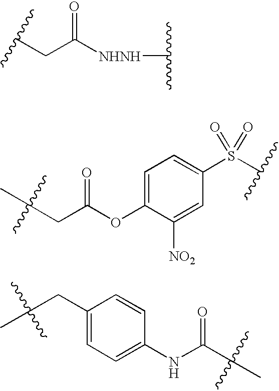

In a particular variation, the group represented by —O—L¹—(L²)ₘ(L³)ₙ— is a moiety selected from the group consisting of substituted or unsubstituted

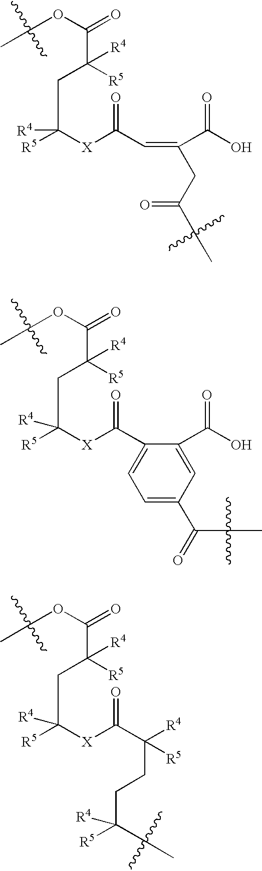

wherein:

X is selected from the group consisting of O, NR^{o}, and S;

R^{o} is hydrogen or substituted or unsubstituted C₁₋₄alkyl and C₁₋₄alkyl CO—; and

each R⁴ and R⁵ is independently selected from the group consisting of hydrogen, hydroxy, C₁₋₄alkyl, C₁₋₄alkoxy and halo, or wherein R⁴ and R⁵ together are oxo. In one variation of the above, each substituent independently comprises 1, 2 or 3 substituents independently selected from the group consisting of halo, hydroxy, cyano, C₁₋₄alkyl, C₁₋₄alkoxy, C₁₋₈alkylamino, C₆₋₁₀aryl and C₄₋₁₂heteroaryl. In another variation of the above compound, Y is S. In a variation of the above, RL—Y— is the N-terminal cysteine sulfhydryl group of cysteine bearing HN-1.

In another variation, the group represented by —X—L¹—(L²)ₘ—(L³)— or —X—L¹— is a substituted or unsubstituted moiety of the formula

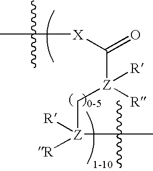

wherein:

each X is independently selected from the group consisting of O, S and NR¹;

each Z is independently selected from N, C, O, and S, provided that the structure does not form two adjacent Z groups that are both N, O or S;

R′ and R″ are each independently selected from the group consisting of H, hydroxy, halo, alkyl, alkyl, aryl, heteroaryl, alkoxy, and amino, each unsubstituted or substituted, or wherein R¹ and R″ together are oxo; and

R¹ is hydrogen or a substituted or unsubstituted C₁₋₄alkyl or C₁₋₄alkylCO—.

In another aspect, there is provided a compound comprising the formulae:

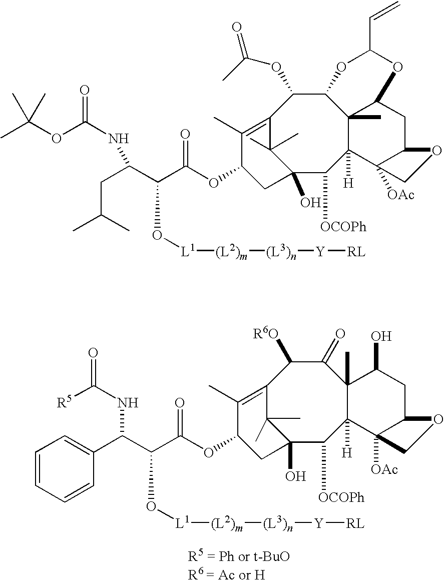

wherein: L¹ is a linker comprising at least 2 atoms in the linker chain, wherein the atoms are selected from the group consisting of C, N, O and S; L² is a linker comprising at least 2 atoms in the linker chain, wherein the atoms are selected from the group consisting of C, N, O and S; L³ is a linker comprising at least 2 atoms in the linker chain, wherein the atoms are selected from the group consisting of C and N; Y is O, NR¹ or S; RL is a ligand; R¹ is hydrogen or a substituted or unsubstituted C₁₋₄alkyl or C₁₋₄alkyl CO—; m is 1, 2, 3, 4 or 5; and n is 0, 1 or 2; or a pharmaceutically acceptable salt or prodrug thereof.

In another aspect, there is provided a compound comprising the formulae:

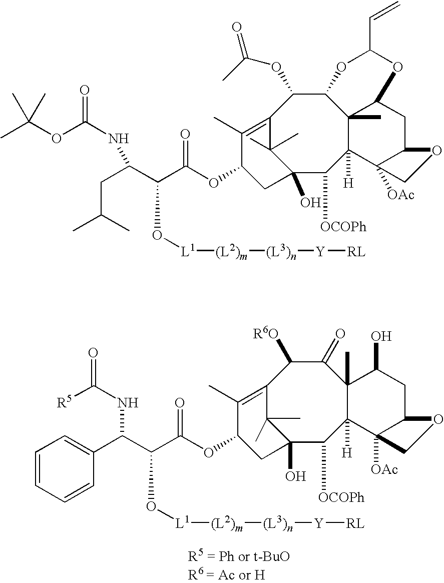

wherein L¹, L² and L³ are each as defined in each aspect and variation above; Y is O, NR¹ or S; RL is a ligand; R¹ is hydrogen or a substituted or unsubstituted C₁₋₄alkyl or C₁₋₄alkyl CO—; m is 1, 2, 3, 4 or 5; and n is 0, 1 or 2; or a pharmaceutically acceptable salt or prodrug thereof

In another aspect, there is provided a compound comprising the formula:

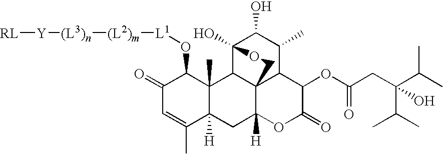

wherein L¹ is a linker comprising at least 2 atoms in the linker chain, wherein the atoms are selected from the group consisting of C, N, O and S; L² is a linker comprising at least 2 atoms in the linker chain, wherein the atoms are selected from the group consisting of C, N, O and S; L³ is a linker comprising at least 2 atoms in the linker chain, wherein the atoms are selected from the group consisting of C and N; Y is O, NR¹ or S; RL is a ligand; R¹ is hydrogen or a substituted or unsubstituted C₁₋₄alkyl or C₁₋₄alkylCO—; m is 1, 2, 3, 4 or 5; and n is 0, 1 or 2; or a pharmaceutically acceptable salt or prodrug thereof.

In another aspect, there is provided a compound comprising the formula:

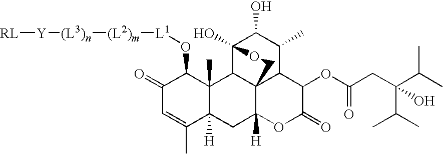

wherein: L¹, L² and L³ are each independently a linker as defined in each of the above variations; Y is O, NR¹ or S; RL is a ligand; R¹ is hydrogen or a substituted or unsubstituted C₁₋₄alkyl or C₁₋₄alkylCO—; m is 1, 2, 3, 4 or 5; and n is 0, 1 or 2; or a pharmaceutically acceptable salt or prodrug thereof.

In yet another aspect, there is provided a compound comprising the formulae:

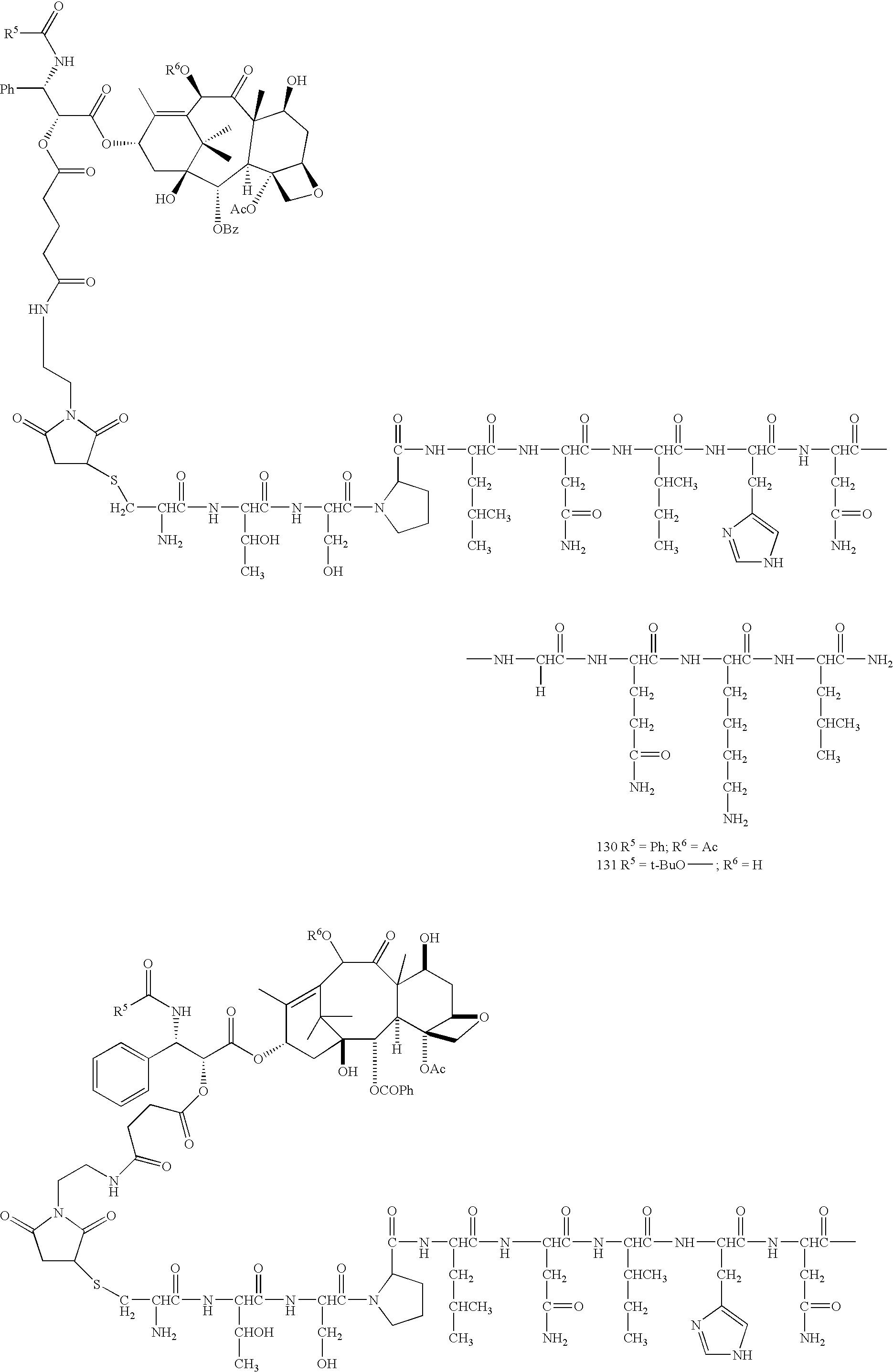

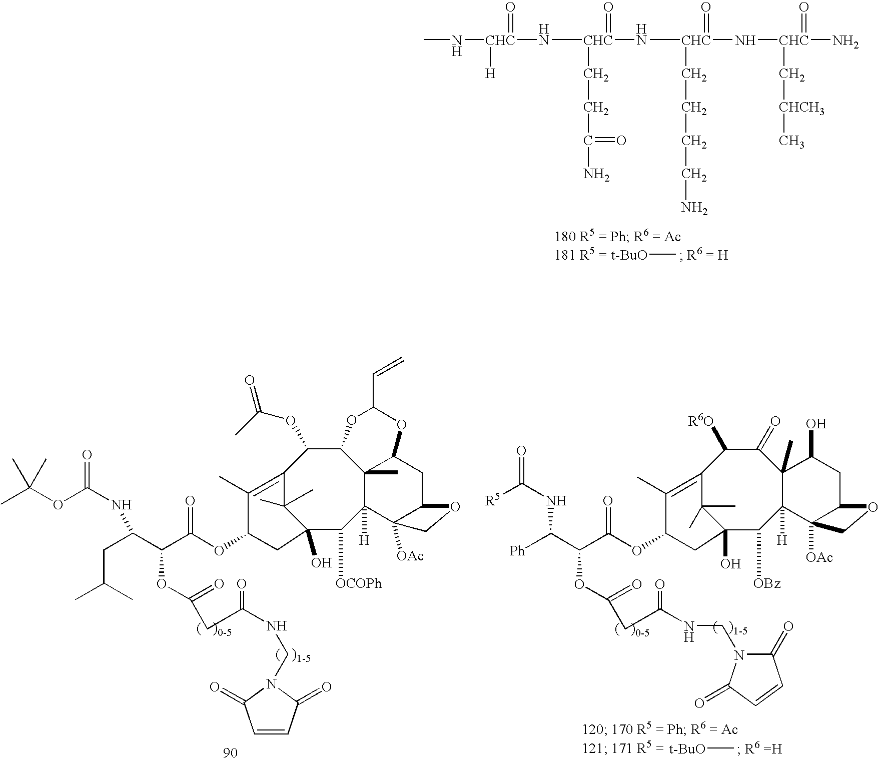

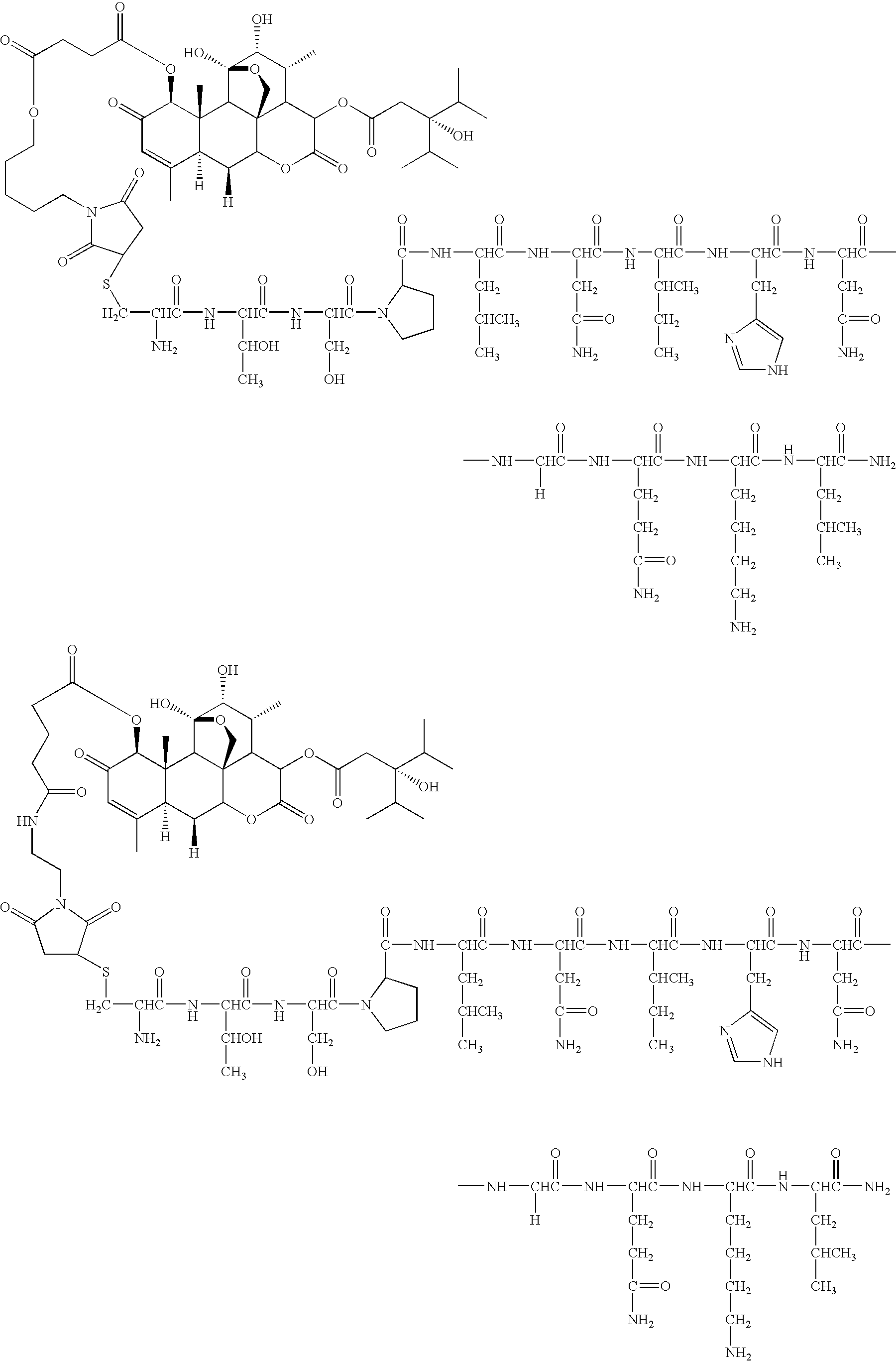

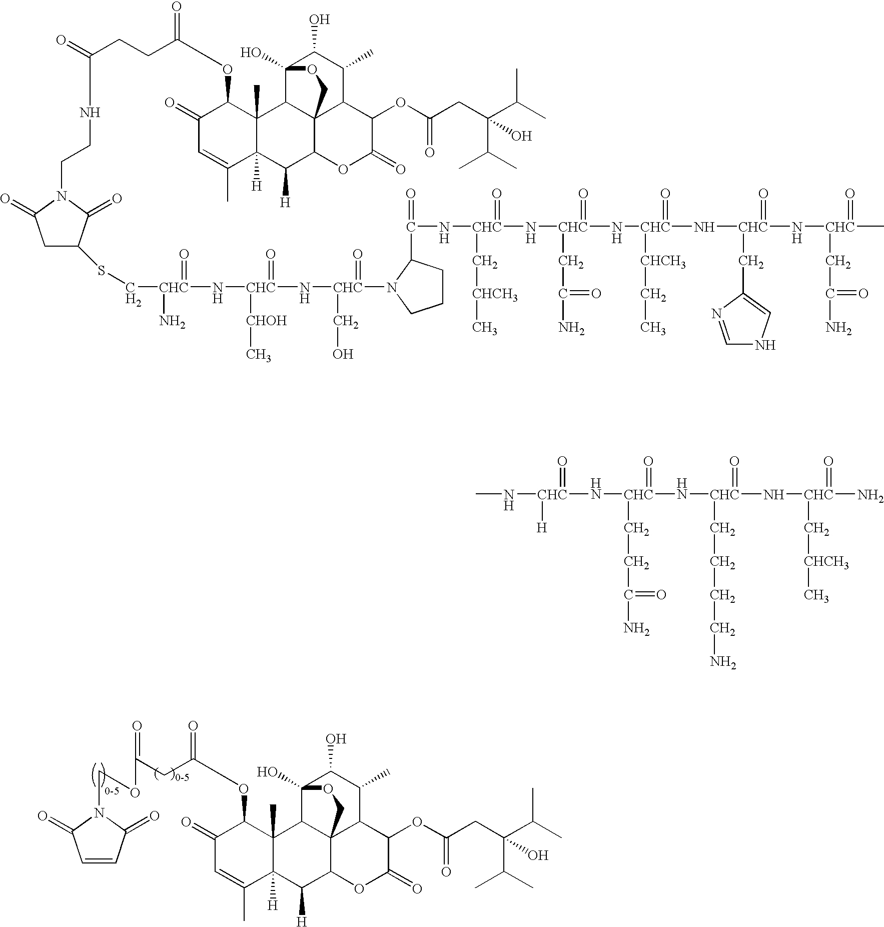

or a pharmaceutically acceptable salt thereof.

In one aspect of the invention, there is provided a pharmaceutical composition comprising, as an active ingredient, a compound of any one of the above. In another aspect, there is provided a method for the treatment or prophylaxis of cancer comprising administration of a therapeutically effective amount of a composition of the above to a patient in need of such a treatment. In another aspect, there is provided a method for the prevention of metastases from tumors comprising administering a composition of the above to a patient in need of such treatment, that is used alone or in combination with radiotherapy and/or other chemotherapeutic treatments conventionally administered to patients for treating cancer. In yet another aspect, there is provided a method for the treatment of cancer by the administration of an effective amount of a composition above, wherein the administration is performed with a chemotherapeutic agent selected from the group consisting of alpha interferon, COMP (cyclophosphamide, vincristine, methotrexate and prednisone), etoposide, mBACOD (methortrexate, bleomycin, doxorubicin, cyclophosphamide, vincristine and dexamethasone), PRO-MACE/MOPP (prednisone, methotrexate, doxorubicin; cyclophosphamide, taxol, etoposide/mechlorethamine, vincristine, prednisone and procarbazine), vincristine, vinblastine, angioinhibins, TNP-470, pentosan polysulfate, platelet factor 4, angiostatin, LM-609, SU-101, CM-101, Techgalan and thalidomide.

In another aspect, there is provided a method for the treatment of cancer by the administration of an effective amount of the above composition, wherein the administration is performed with a chemotherapeutic agent selected from the group consisting of alkylating agents, nitrogen mustards, mechloethamine, melphan, chlorambucil, cyclophosphamide and ifosfamide; nitrosoureas including carmustine, lomustine, semustine, streptozocin; alkyl sulfonates, busulfan; triazines, dacarbazine; ethyenimines, thiotepa, hexamethylmelamine; folic acid analogs, methotrexate; pyrimidine analogues, 5-fluorouracil, cytosine arabinoside; purine analogs, 6-mercaptopurine, 6-thioguanine; antitumor antibiotics, actinomycin D; anthracyclines, doxorubicin, bleomycin, mitomycin C, methramycin; hormones and hormone antagonists, tamoxifen, cortiosteroids; cisplatin and brequinar.

In one aspect, there is provided a method of treating a patient suffering with cancer comprising administering to the patient: (i) a first component consisting of pharmaceutical composition comprising as an active ingredient a compound of formula I or formula III:

B—X—L¹—(L²)ₘ—(L³)ₙ—Y—RL I

TQ—O—L¹—(L²)ₘ—(L³)ₙ—Y—RL III

wherein: B is a biologically active agent, analogs and derivatives thereof; TQ is a taxane or a quassinoid derivative; L¹ is a linker comprising at least 2 atoms in the linker chain, wherein the atoms are selected from the group consisting of C, N, O and S; L² is a linker comprising at least 2 atoms in the linker chain, wherein the atoms are selected from the group consisting of C, N, O and S; L³ is a linker comprising at least 2 atoms in the linker chain, wherein the atoms are selected from the group consisting of C and N; X and Y are each independently O, NR¹ or S; RL is a ligand; R¹ is hydrogen or a substituted or unsubstituted C₁₋₄alkyl or C₁₋₄alkylCO—; m is 1, 2, 3, 4 or 5; and n is 0, 1 or 2; or a pharmaceutically acceptable salt or prodrug of the compound; wherein the compound of formula I is administered in an amount of from about 10 mg/m² per day to about 500 mg/m² per day for up to about 14 days starting on the first day of a 28 day cycle, and (ii) a second component consisting of an injection solution comprising as an active ingredient a chemotherapeutic agent which is administered in amount of from about 10 mg/M² to about 500 mg/m² on the first day of a 28 day cycle, the 28 day cycle being repeated as long as the tumor remains under control. In one variation, the compound of formula I is administered in an amount of about 10 mg/m² per day to about 400 mg/m² per day, in an amount of about 10 mg/m² per day to about 300 mg/m² per day, in an amount of about 10 mg/m² per day to about 200 mg/m² per day, or in an amount of about 10 mg/m² per day to about 100 mg/m² per day. In another variation, the compound of formula I is administered in an amount of about 100 mg/m² per day to about 500 mg/m² per day, in an amount of about 200 mg/m² per day to about 500 mg/m² per day, in an amount of about 300 mg/M² per day to about 500 mg/m² per day, or in an amount of about 400 mg/m² per day to about 500 mg/m² per day. In a particular variation for each of the above dosage amount, the compound is administered in the dosage amount per day for up to about the third, seventh, tenth or 14 days starting on the first day of a 28 day cycle, and the second component is administered in one of the permutations of the above dosages and day cycles as deemed to be effective for the particular cancer and/or patient. In one variation of the above method, the cancer is selected from the group consisting of breast cancer, ovarian cancer, colon cancer, pancreatic cancer, prostate cancer, gastric cancer, lymphoma, leukemia, skin carcinoma, lung carcinoma, head and neck carcinoma. In another variation, the pharmaceutical composition is administered by the means of injection or intravenous infusion. In each of the above embodiments, aspects and variations, the ligand is a receptor ligand. In another aspect of each of the above, the ligand is a peptide, a protein or a targeting peptide with internalization or without internalization.

The therapeutic conjugates of the present invention may be administered with a second therapeutic agent to an animal or human in a sequential manner. Generally, the conjugate and the second therapeutic agent may be administered at times effectively spaced apart to allow the conjugate and the second agent to exert their respective therapeutic effects. For example, the conjugate may be administered to the animal or human at a time prior to the second therapeutic agent, or at a time subsequent to the conjugate of the present invention. Such methods and considerations will be known to one skilled in the art. Such methods and protocols for providing combination cancer therapies have been previously disclosed, for example, in U.S. Pat. No. 6,548,531 which is incorporated herein by reference.

For each embodiments, aspects and variations disclosed in the present invention, the compounds of the invention covers all pharmaceutically acceptable ionized forms, such as salts, and solvates of the compounds even if the ionized forms and solvates are not explicitly noted, as it is well known in the art to administer pharmaceutical agents in an ionized or solvated form. Also, unless a particular stereochemistry is specified, recitation or graphical representation of a compound is intended to cover all possible stereoisomers, that is, enantiomers and diastereomers; and all resonance forms and their tautomers.

In each of the above aspects of the disclosure as recited herein, including all aspects, embodiments and variations and representative examples, are intended to be interchangeable where applicable, such that the various aspects, embodiments and variations may be combined interchangeably and in different permutations. For example, the compound of formula I, II or III comprising a biologically active agent, a ligand and a linker L may each be combined in different permutations and variations, such that, for example, a particular biologically active agent may be combined with any one particular ligand and any one particular linker to form the compound of the present disclosure. Accordingly, a linker such as L¹ that is present in the above formulae, for example, may be a substituted or unsubstituted dicarbonyl compound comprising a cyclic system, an acyclic system, or the linker may be a disulfoxide, a sulfone, or sulfonamide linker and their various permutations as disclosed in the application because the linker is a bivalent linker and may be readily exchanged with any other bivalent linker.

A number of alternative modifications, embodiments and aspects of the invention will be apparent to one skilled in the art to which this disclosure pertains having the benefit of the teachings presented in the disclosure. Accordingly, it is to be understood that the invention is not to be limited to the specific embodiments or aspects disclosed and that modifications and other embodiments and aspects are intended to be included within the scope of the invention. Although specific terms are employed in the present application, they are used in a generic and descriptive sense as understood to one skilled in the art only, and the terms are not for purposes of limitation.

### Preparation of Molecular Conjugates:

A general, representative scheme showing one convergent method for the preparation of molecular constructs that are suitable for peptide conjugate with a biologically active compound, such as a taxane derivative, is shown below. TAX—OH, a taxane derivative or analogue with a free hydroxyl group, may be condensed with a linker derivative (“L¹”) to form the Taxane-Linker1 compound. The free hydroxyl group may be in any available position on the taxane derivative, including the 2′, 7 or 10 position. Independently, a “P²” protected linker L² may be coupled with linker L³ to form the L²-L³ linker. The protecting group P² and any other protecting groups, as deemed necessary, may be removed prior to the subsequent coupling reaction with the Taxane-Linker1. The L²-L³ linker may be condensed or coupled with the Taxane-Linker1 to form the conjugate compound represented in the Scheme. As shown herein, the linkers (L¹, L² and L³) are divalent groups represented with a left end and a right end for linking with adjacent groups. However, it is intended that with asymmetric linkers, the left end and right ends of the linkers may be used reversibly and interchangeably, depending on the nature of the linking group, the protecting groups and/or the group to which the linkers are linked to. The use of protecting groups prior to a condensation or coupling reaction and the removal of the protecting group after the reaction will be employed as deemed necessary, depending on the nature of the coupling reagents, the linking groups, and the reaction conditions employed as is known to one skilled in the art of organic synthesis.

As shown in the general Scheme, the coupling or condensation reactions may be performed using a convergent strategy, a linear strategy or the combination of both as deemed most efficient, depending on the nature and availability of the reactants, the linking groups, the reaction conditions, and the efficiency of the reactions.

Protecting groups that may be used may include those known in the art and or described in standard texts such as, for example, in T. W. Greene, Protecting Groups in Organic Synthesis, 3rd edition, John Wiley & Sons, Inc. 1999. Hydroxyl protecting groups may include, for example, those derived from silanes such as tert-butyl dimethylsilyl groups (TBDMS) or their various analogues, and removal of silyl ether protecting groups may be performed using fluoride or under acidic conditions, such as mild organic acids in aqueous, organic protic or aprotic solvent or mixtures thereof. As used herein, a protecting group may be employed during a reaction transformation and may be removed from the desired product, or the “protecting group” may also constitute an element or component of the final active conjugate.

The reactions may be performed in an inert atmosphere in an organic solvent or mixture of solvents, and when necessary, may also include the addition of water. The reactions may be performed at a temperature range of about −78° C. up to refluxing temperature of the solvent or solvent mixtures. Isolation and purification of the product from the product mixture may be accomplished using various procedures known in the art of organic synthesis.

### SCHEME: General Procedure for the Preparation of a Taxane Peptide Conjugates:

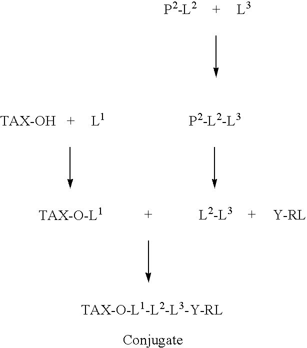

More specifically, the preparation of the conjugate of a taxane, such as paclitaxel, docetaxel or compound 70 and a peptide may be prepared by synthesizing the linker, attaching the linker to taxane, coupling the peptide to the linker-taxane construct and finally, purifying the conjugate. The conjugate may be further converted to the corresponding salt. Related methods for the preparation of Transferrin-drug conjugates for use in cancer treatment is disclosed in U.S. Pat. No. 6,825,166, the disclosure of which is incorporated herein by reference.

Representative procedures for forming various taxanes are described in U.S. Pat. Nos. 5,675,025; 5,684,175; 5,770,745; 5,939,566; 5,948,919; 6,048,990; 6,066,749; 6,072,060; 6,136,999; 6,143,902; 6,262,281; 6,307,088; 5,688,977 and 6,107,497, the disclosures of which are incorporated herein by reference.

Pharmaceutical compositions for administration of the compounds of the present invention may also contain minor amounts of auxiliary substances such as wetting agents, emulsifying agents, or solubilizing agents, pH buffering agents and the like, including for example, acetate, sodium citrate, cyclodextrin derivatives, and other related agents. Other methods of preparing the dosage forms are known in the art or are apparent to those skilled in this art. Example of a reference include “Remington: The Science and Practice of Pharmacy”, A. Gennaro, ed., 20th edition, Lippincott, Williams & Wilkins, Philadelphia, Pa.

### Equipment and Materials:

HPLC/LCMS: HPLC/LCMS analyses were performed on Hewlett Packard (HP) series 1100 instruments with UV detection (Hewlett Packard Corp., Palo Alto, Calif.). HPLC methods used were: Method 1: Column: Phenomenex Luna (C18) 2 mm; Column Flow: 0.5 mL/min; Stop Time: 14.00 min; Solvents: 0-100% H₂O/ACN; Inj. Vol: 5 μL; m/z: 500-4000, target 2500; Column Temp: 40.0° C.; Method 2: Column: Luna mercury (C18) 2 mm; Column Flow: 0.5 mL/min; Stop Time: 7.00 min; Solvents: 0-100%; 0.01% Formic Acid/H₂O/ ACN; Inj. Vol: 1 μL; m/z: 500-4000, target 2500; Column Temp: 40.0° C.; Method 3: Column: Phenomenex Proteo 15 cm×4.6 mm×4 μm; Column Flow: 1.0 mL/min; Stop Time: 58 min; Solvents: 0.1% TFA/H₂O/ ACN (12—45-95% ; 0—22—32—47 min); Inj. Vol: 20 μL; Column Temp: 40.0° C.; Method 4: Column: Proteo 150×4.6 mm×4 μm; Column Flow: 1.0 mL/min; Stop Time: 12 min; Solvents: 0.1% TFA/H₂O/ACN; Inj. Vol: 10 μL; Column Temp: 35.0° C.; Method 5: Column: Proteo 250×4.6 mm×4 um; Column Flow: 1.5 mL/min; Stop Time: 16 min; Solvents: 0.1% TFA/H₂O/ACN; Inj. Vol: 10 L; Column Temp: 35.0° C.

Analytical TLC plates: EM Science (EM Industries, Inc., Gibbstown, N.J.) Silica Gel 60 F₂₅₄, precoated aluminum sheets, Cat. No.5554/7, 200 μm. TLC and PLC plates were eluted, allowed to air dry and visualized under UV-254 light and/or iodine. SPE Cartridges: Bakerbond SPE Octadecyl (C18), JT Baker, Cat. No. 7020-07, (Mallinckrodt Baker, Inc., Phillipsburg, N.J.). GC/MS: Agilent 6890N with 5973 MSD (Agilent, Palo Alto, Calif.), analyses were performed on the GEN method. LC/MS: Analyses were performed on either the Thermo Quest Finnegan LCQ Deca (San Jose, Calif.) or the Agilent 1100 Series LC/MSD Trap (Palo Alto, Calif.) using HPLC methods mentioned above.

Reagents were obtained from Aldrich Chemical Co., Milwaukee, Wis.; purities are all >98% unless otherwise indicated. Solvents were obtained from Burdick & Jackson, Muskegon, Mich. and EM Science, Gibbstown, N.J., and Van Waters & Rogers, Inc., Kirkland, Wash.

### Abbreviations and Definitions:

ACN=acetonitrile
Brine=saturated aqueous sodium chloride solution
DCC=1,3-dicyclohexylcarbodiimide
DCM=dichloromethane
DCU=dicyclohexylurea
DEAD=diethyl azidodicarboxylate
DMAP=4-dimethylaminopyridine
DMF=N,N-dimethylformamide
EtOAc=ethyl acetate
g=gram(s)
GC/MS=gas chromatography with mass spectrometry
HF-pyridine=hydrogen fluoride pyridine
LCMS=liquid chromatography with mass spectrometry
mg=milligram(s)
mL=milliliter(s)
mmol=millimole(s)
MTBE=methyl-t-butyl ether
NaHCO₃=sodium bicarbonate
Na₂SO₄=sodium sulfate
N₂=dry nitrogen gas
rt=room temperature
TBDMS=t-butyldimethylsilyloxy
THF=tetrahydrofuran
TPP=triphenyl phosphine
TPPO=triphenyl phosphine oxide

The preparation of an alternatively linked paclitaxel- HN1 peptide construct, 60 is exemplified below:

5-TBDMS-1-pentandiol, 10 was converted to a protected maleimide, 5-TBDMS-1-(2,5 dioxo-2,5-dihydro-pyrrol-1-yl)pentane, 20. The TBDMS group was then removed to produce the alcohol-maleimide derivative, 30. Compound 30 was condensed to paclitaxel-2′-hemisuccinate, 40, to form the paclitaxel maleimide derivative, 50, suitable for conjugation to sulfhydryl containing peptides. Compound 50 was conjugated to the N-terminal cysteine sulfhydryl group of cysteine bearing HN-1 peptide to form the peptide-linker-paclitaxel conjugate, 60.

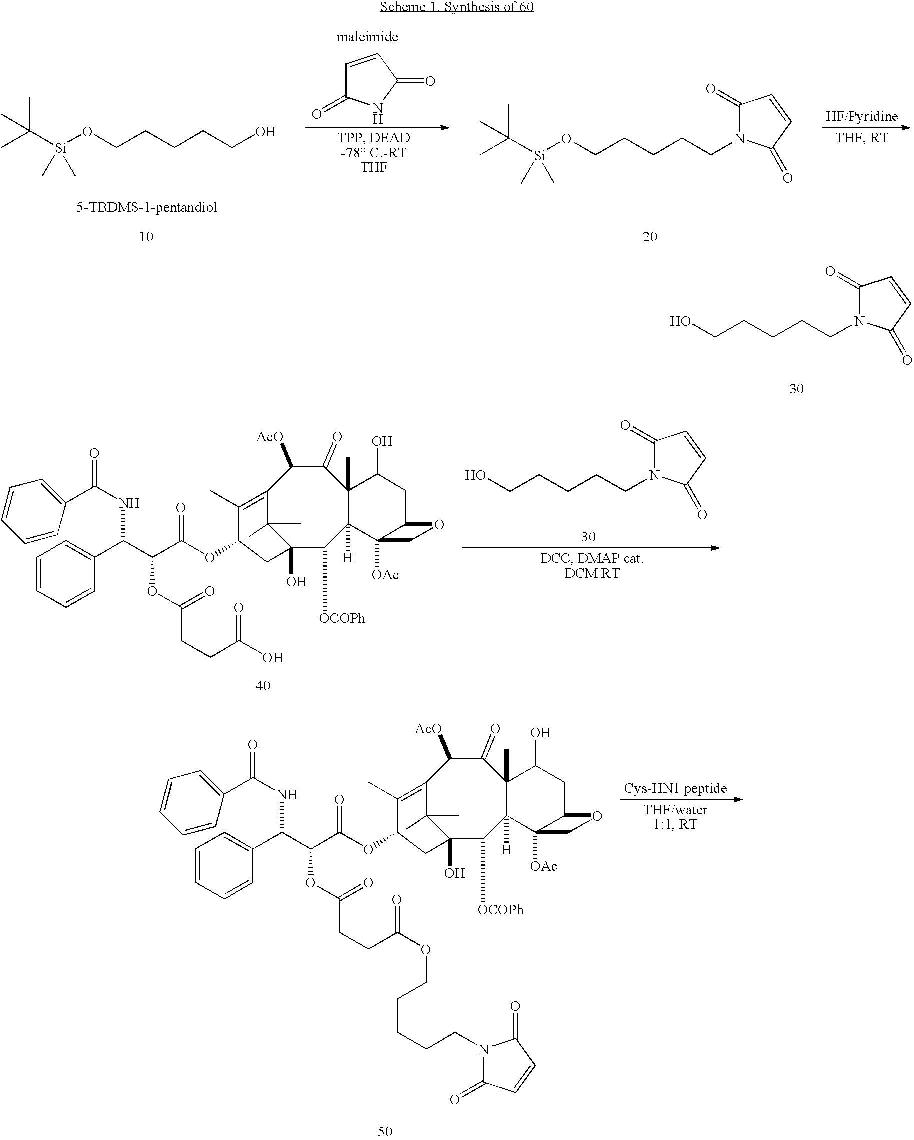

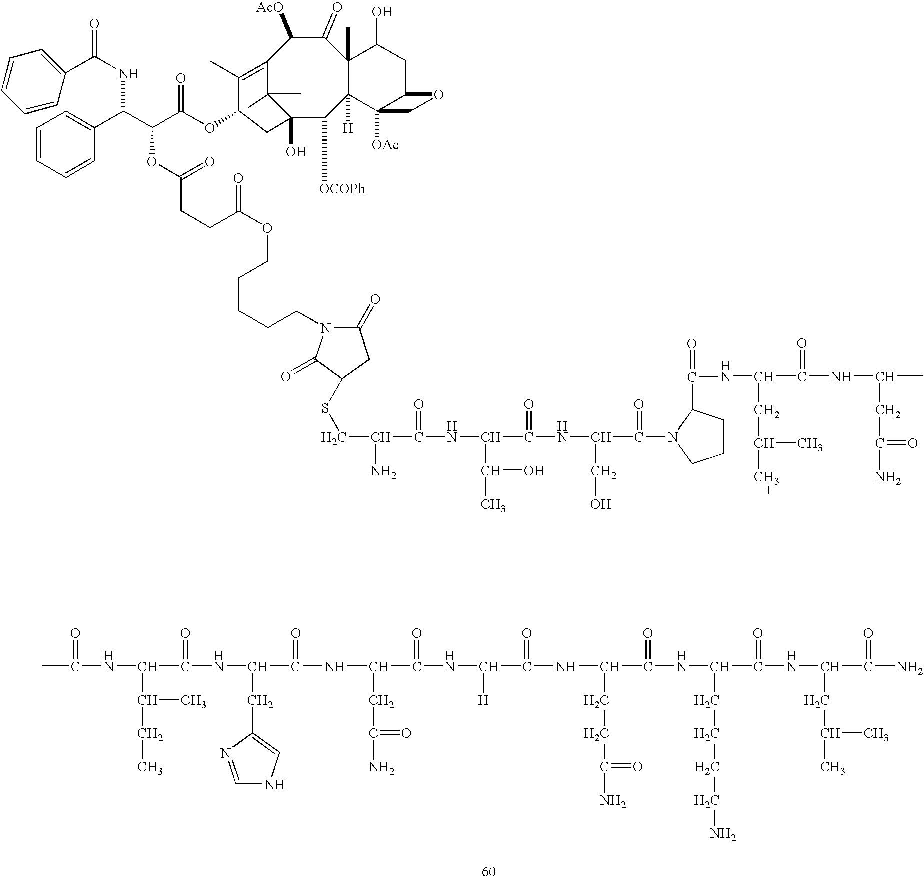

### Procedures:

Synthesis of N-(5-O-TBDMS pentyl)-maleimide 20:

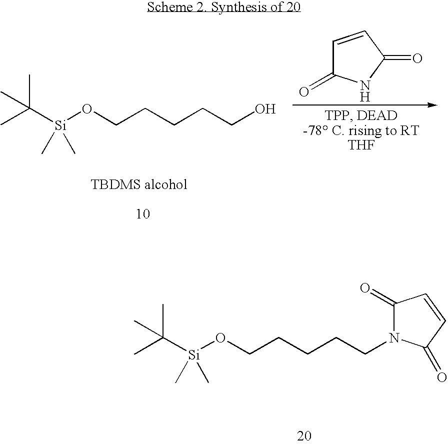

TBDMS alcohol 10 (2.18 g, 10.0 mmol) was weighed into a 200 mL round bottomed flask. To this was added THF (50 mL) and stirring was begun under N₂ with cooling to −78° C. (acetone-dry ice). TPP (2.62 g, 10.0 mmol), in solution with 15 mL of THF, was added at −78° C. This was followed by the addition of DEAD (1.84 g, 10.0 mmol) and maleimide (0.97 g, 10.0 mmol), solids added neat. The reaction mixture was stirred for 5 min. at −78° C., allowed to rise to room temperature and was stirred overnight. GC/MS indicated conversion to the product had occurred. The reaction mixture was concentrated to a residue on the rotavap and 50 mL MTBE was added to precipitate TPPO. The TPPO solids were filtered and the filtrate was concentrated to an oily residue, which was purified by silica gel column chromatography with 3:1 heptane/EtOAc to give the TBDMS maleimide, 20.

N-(5-hydroxypentyl)-maleimide 30:

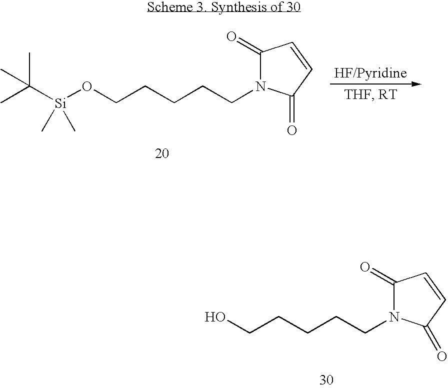

Compound 20 (0.594 g, 2.0 mmol) was weighed into a 50 mL round bottomed flask and dissolved in 5 mL of THF at room temperature under N₂ with stirring. HF-Pyridine (2 mL) was added and the reaction mixture was allowed to stir at room temperature. After 6 hours, HPLC indicated that the reaction had gone to completion. The solvents were evaporated on the rotavap at reduced pressure with a bath temperature of 25° C. EtOAc (25 mL) was added and the mixture was transferred to a separatory funnel and washed with 25 mL of 10% NaHCO₃, 25 mL of water and 25 mL of brine. The organic phase was dried over Na₂SO₄ and concentrated. The product was purified by flash chromatography on a silica column with elution with 3:1 heptane/EtOAc to give the alcohol, 30, used without further purification in the next step. Paclitaxel-2′ succinyl-5-maleimido pentanoate 50:

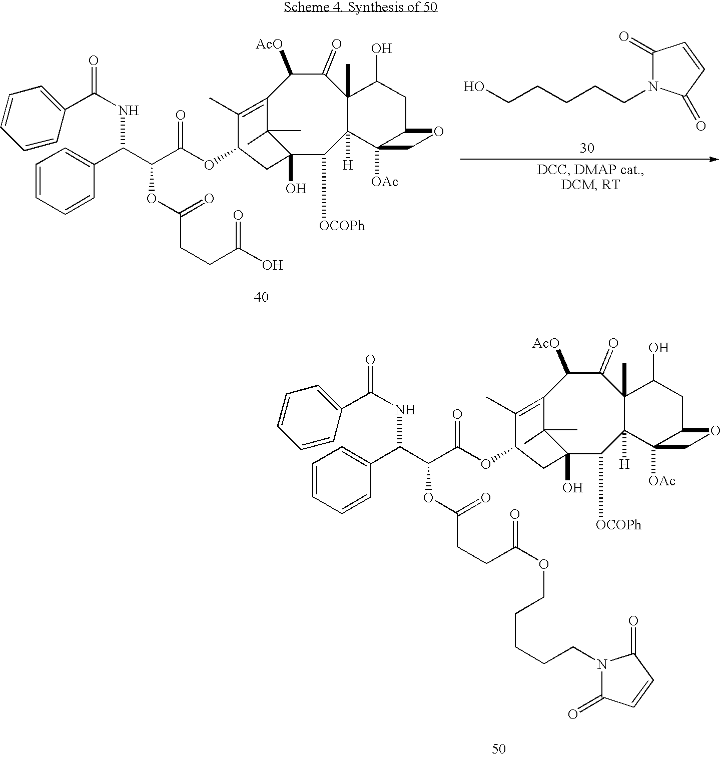

The maleimide alcohol derivative, 30 (0.183 g, 1.0 mmol), paclitaxel—2′-hemisuccinate, 40 (0.953 g, 1.0 mmol), and DMAP (0.122 g, 1.0 mmol) were weighed into a 25 mL round bottom flask. Dry DCM (10 mL) was added with stirring under N₂. DCC (0.206 g, 1.0 mmol) was added and the reaction stirred overnight. The DCM was evaporated; 50 mL of MTBE was added; and the precipitated DCU was filtered. The concentrated filtrate was purified by flash chromatography on silica gel with elution with 60:40 EtOAc/heptane to give 600 mg of product, 50 homogeneous by HPLC.

Synthesis of a paclitaxel peptide conjugate, 60:

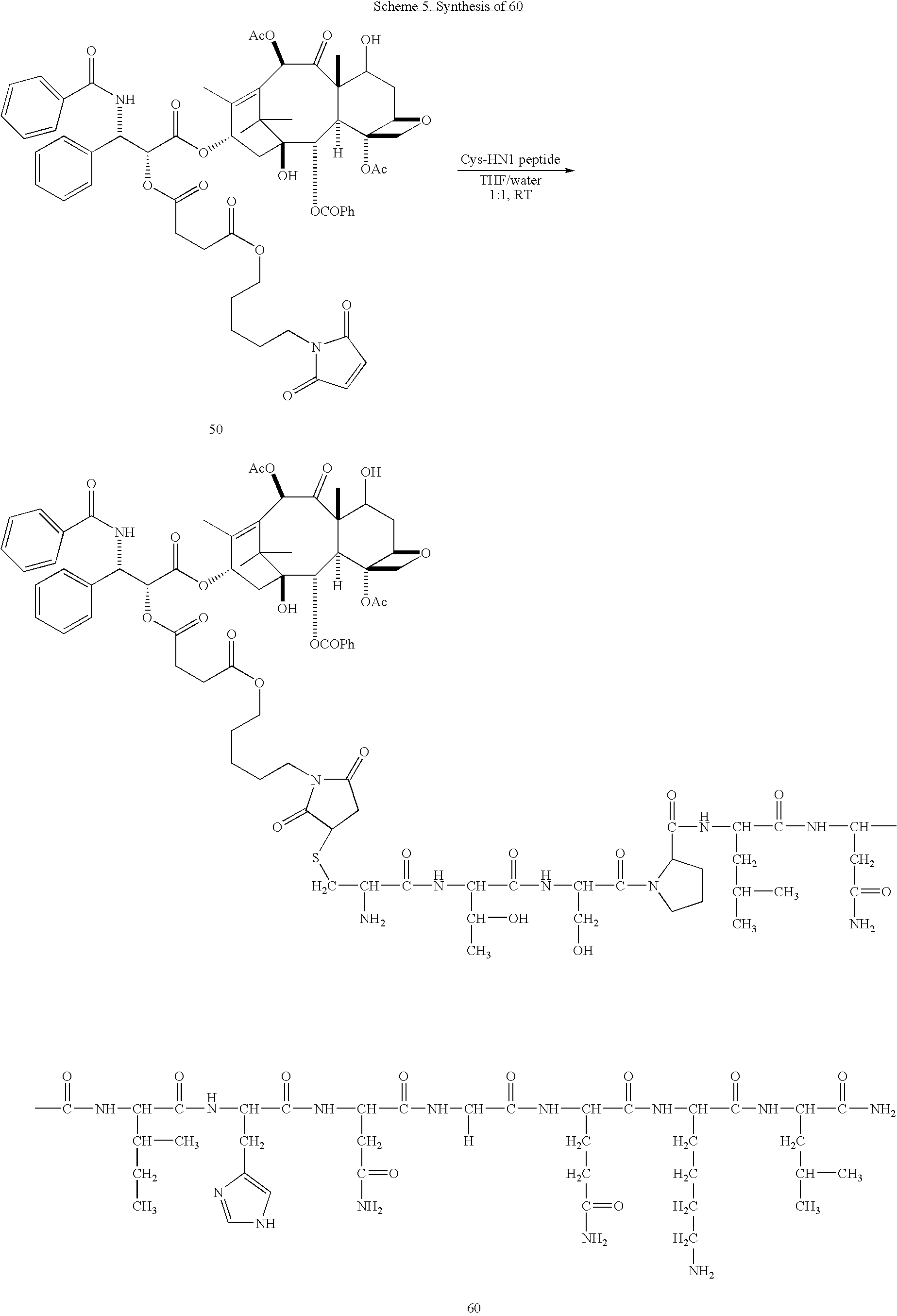

The peptide, Cys-HN-1 (0.014 g, ˜0.01 mmol, Global Peptide, Ft. Collins, Colo.), was weighed into a 10 mL pear shaped flask. Distilled water (1 mL) was added followed by 2 mL of a solution of the paclitaxel-2′ succinyl-5-maleimido pentanoate, 50, in THF (11 mg /10 mL THF). The mixture was stirred at room temperature. After 2 hours, characterization by LC/MS (method 1) showed a product with MW 2540, corresponding to the conjugate 60. The solvents were evaporated on the rotavap at reduced pressure to a slurry. This mixture was dissolved in water with a small amount of added acetonitrile and applied to a solid phase separation cartridge (Sep Pak C-18): elution with 90:10, 80:20, 70:30, and 50:50 water/acetonitrile in 5 mL portions. The product, 60, eluted with the 70:30 fraction. It was concentrated and dried on high vacuum, yielding approximately 8 mg of the product homogeneous by HPLC. Conversion of Taxane 70 to its 2′-Hemisuccinate Taxane compound 80:

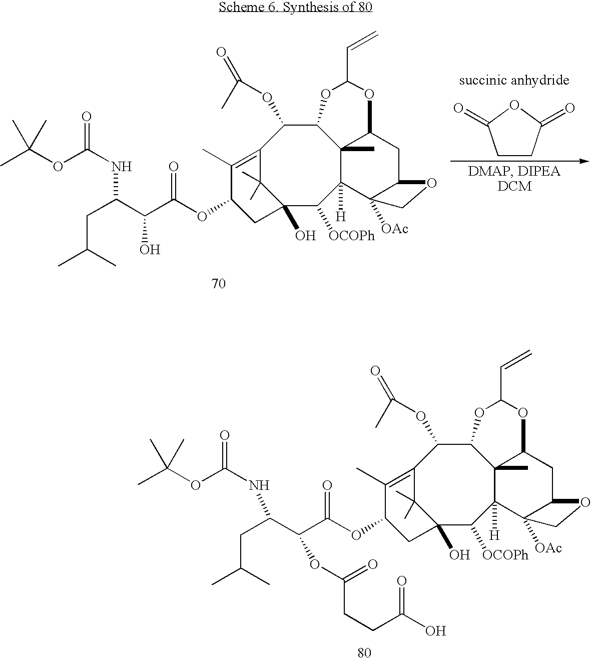

To compound 70 in DCM (0.5 mL) was added succinic anhydride (20 mg, 1.7 eq), DIPEA (45 μL, 2.2 eq) and DMAP (5 mg, 0.36 eq). The reaction mixture was stirred for 1.5 hr when no starting material was observed by LCMS. The reaction was quenched with 10% citric acid solution (0.5 mL), then diluted with DCM (5 mL) and water (5 mL) and partitioned. The organic layer was washed with brine (5 mL) and water (5 mL), dried over magnesium sulfate and concentrated to dryness to give 0.1029 g of the compound 80.

### Conversion of 2′-Hemisuccinate Taxane 80 to 2′-Succinamidoethylmaleimide Taxane Compound 90:

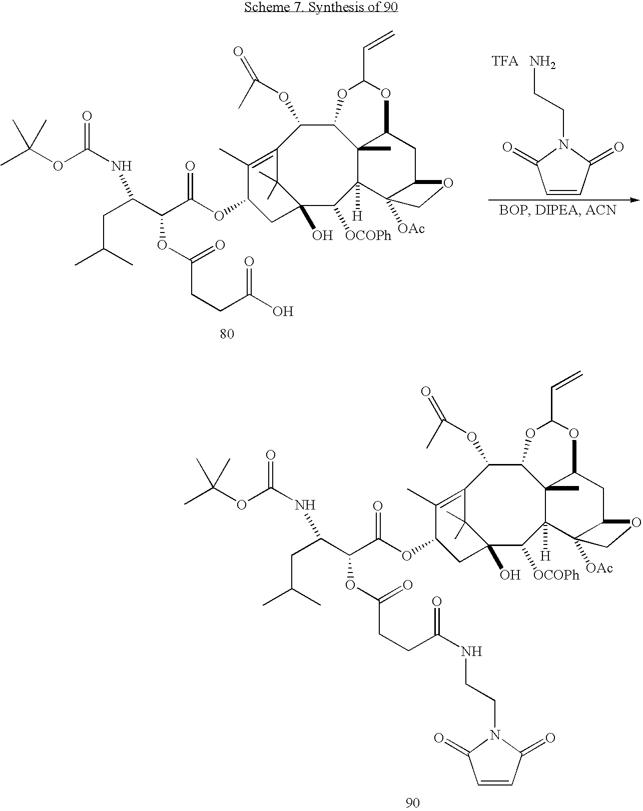

To the succinate derivative 80 (100 mg) in acetonitrile (1 mL) was added DIPEA (40 μL, 2 eq), and BOP reagent (115 mg). LC/MS indicated no starting material present at the first sample point (˜5 min). Two intermediates had formed. N-(2-Aminoethyl)maleimide trifluoroacetic acid salt (32 mg, 1.2 eq, Toronto Research chemicals, Inc.) and DIPEA (20 μL, 1 eq) were added to the reaction mixture, and after 1 hr no intermediates were observed by LC/MS. The reaction mixture was partitioned with IPAc and water. It was then purified by a silica plug, concentrated and dried to give 0.0736 g of the succinate—maleimide derivative 90.

### Synthesis of Taxane Peptide Conjugate 100:

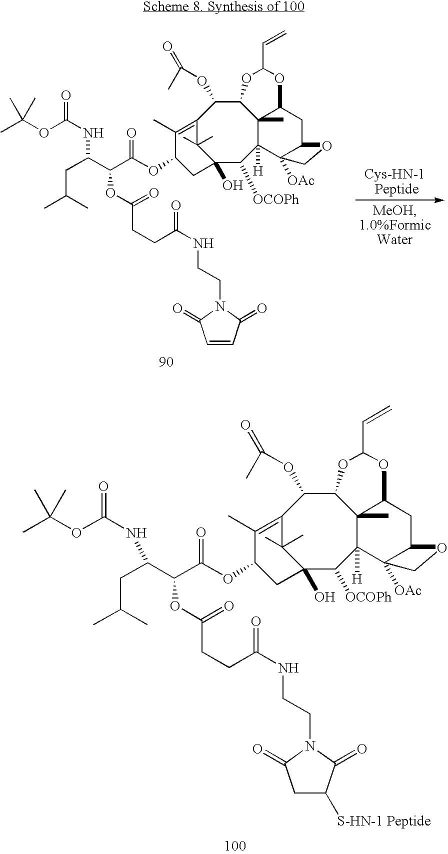

A solution of the Cys-HN-1 peptide (1.30 g, 0.913 mmol) in milli-Q water (8.0 mL) was added to a solution of 90 (0.630 g, 0.5768 mmol) in tetrahydrofuran (24.0 mL) at room temperature and stirred for 3 hr when it went to completion as indicated by LC/MS (Method-2). The reaction mixture was evaporated to dryness and the residue was dissolved in 5% acetonitrile in water 10 mL water with sonication and loaded on a reverse phase column (C-18). The unreacted peptide was washed out of the column with 15% CH₃CN in water. The product was eluted out of the column with 50% CH₃CN in water. The fractions were analyzed by LC-MS (Method-2) and the pure fractions were pooled, concentrated and the residue was lyophilized to give the pure product 100.

Synthesis of Paclitaxel-2′-hemiglutarate 110:

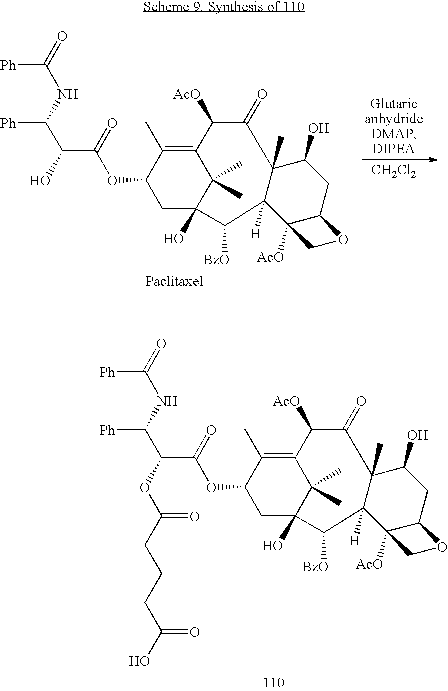

Paclitaxel (5.0 g, 5.855 mmol), DMAP (0.143 g, 1.17 mmol), glutaric anhydride (0.735 g, 6.44 mmol) and anhydrous CH₂Cl₂ (60 mL) were added sequentially to a round-bottomed flask at room temperature under a nitrogen blanket and stirred. DIPEA (2.24 mL, 12.88 mmol) was then added to the reaction mixture, at room temperature, dropwise over five minutes via a syringe. The reaction mixture was stirred at room temperature for 3 hours when it went to completion as shown by TLC (wet acidified IBAC, i.e. 1% H₂O, 1% AcOH in IBAC). The reaction mixture was diluted with CH₂Cl₂ (50 mL) and was quenched with 10% w/v aqueous citric acid solution (30 mL). The mixture was stirred for 15 minutes and the organic layer separated. The organic layer was washed with water (25 mL), brine (30 mL), dried over MgSO₄ (10 g) and roto—stripped to give 6.5 g of a white solid. The white solid was recrystallized from acetone and water. The product was filtered, washed with water (10 mL) and dried overnight in a vacuum oven to give 5.28 g of 110 (93.16% theoretical yield). Melting point=190-192° C. Conversion of Paclitaxel-2′-hemiglutarate 110 to its aminoethylmaleimide derivative 120:

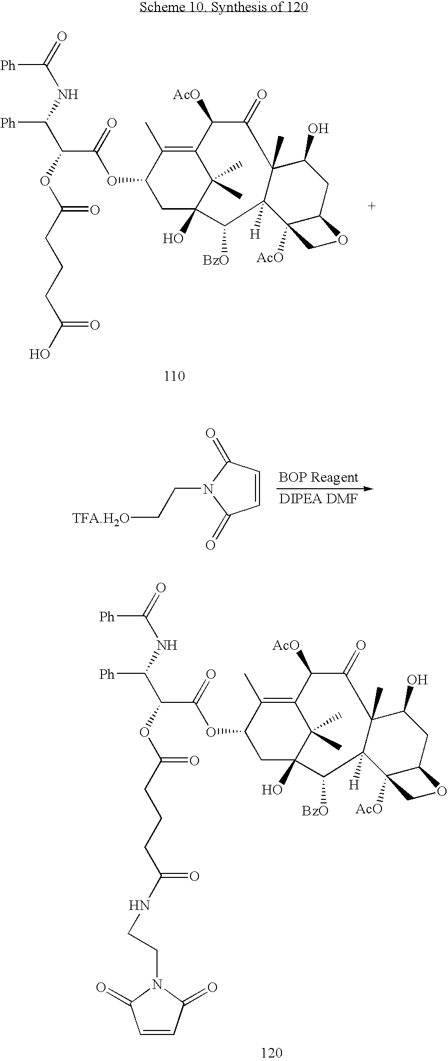

N—(2—aminoethyl)—maleimide TFA salt (0.131 g, 0.516 mmol), DIPEA (0.18 mL, 1.032 mmol) and BOP reagent (0.230 g, 0.516 mmol) were added sequentially to a solution of 110 (0.500 g, 0.516 mmol) in anhydrous DMF (2.5 mL) at room temperature under nitrogen atmosphere. The reaction mixture was stirred overnight at room temperature. The reaction was quenched by the addition of water (5 mL) and the mixture was extracted with ethyl acetate (30 mL). The ethyl acetate layer was washed with water (2×20 mL), brine (1×10 mL), dried (MgSO₄, 5 g) and roto—stripped to give the crude product, which was purified by a silica plug to give pure maleimide derivative 120.

### Synthesis of Paclitaxel Peptide Conjugate 130:

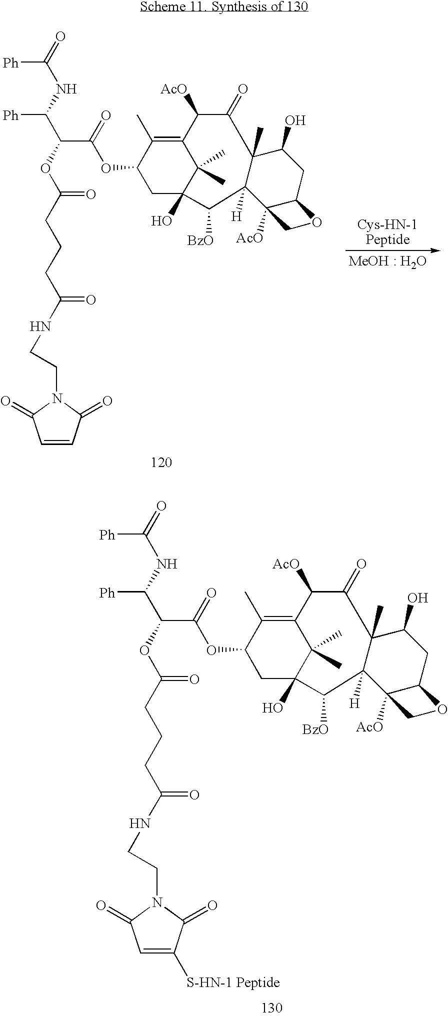

A solution of the Cys—HN-1 peptide (0.0326 g, 0.0229 mmol) in milli—Q water (0.35 mL) was added to a solution of 120 (0.025 g, 0.0229 mmol) in 1% formic acid in methanol (0.75 mL) at room temperature and stirred for 2 hr when it went to completion as indicated by LC/MS. The reaction mixture was evaporated to dryness and the residue was dissolved in 1 mL of water with sonication and loaded onto an SPE cartridge (C-18). The cartridge was washed with two column volumes of water to remove any unreacted peptide and then with 25% CH₃CN in water containing 0.1% formic acid (three column volumes). The fractions were analyzed by HPLC (method 3) and the pure fractions were pooled, concentrated and the residue was dissolved in 1.5 mL water with sonication. The solution was lyophilized to give 39 mg white flaky solid 130. HPLC area %=99.3%.

The same experimental conditions were used to conjugate the Cys-HN-J peptide with the 2′glutarylmaleimido and 2′succinylmaleimido paclitaxel derivatives to afford the HN-J analog of 130 and the HN-J analog of 180 respectively.

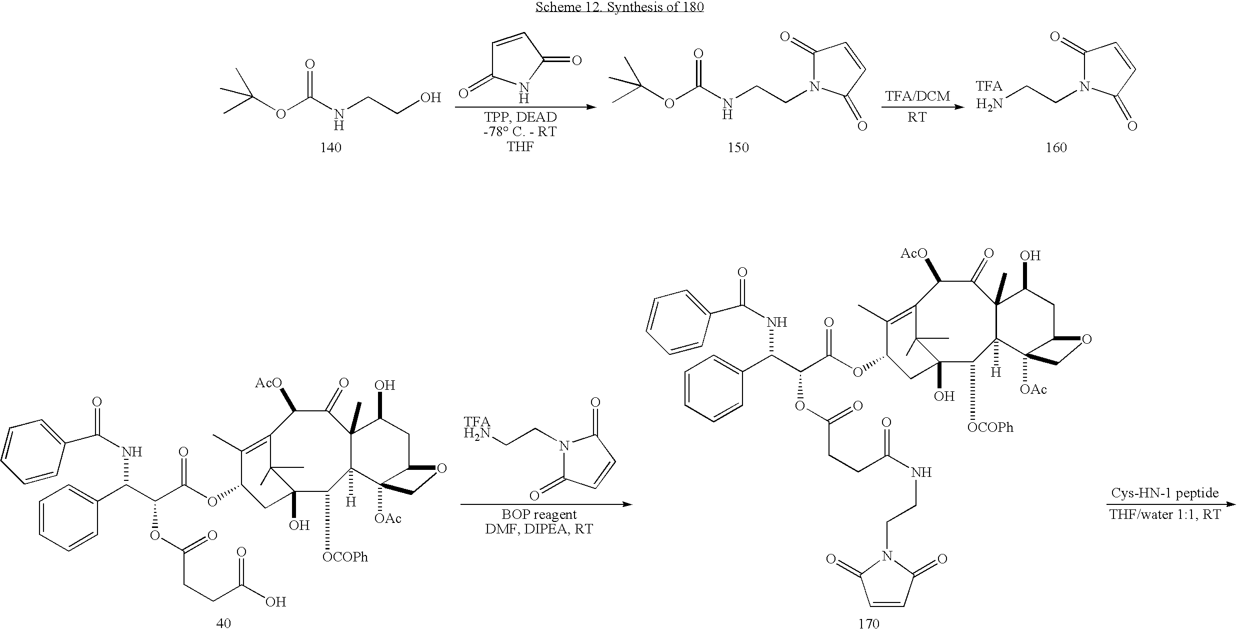

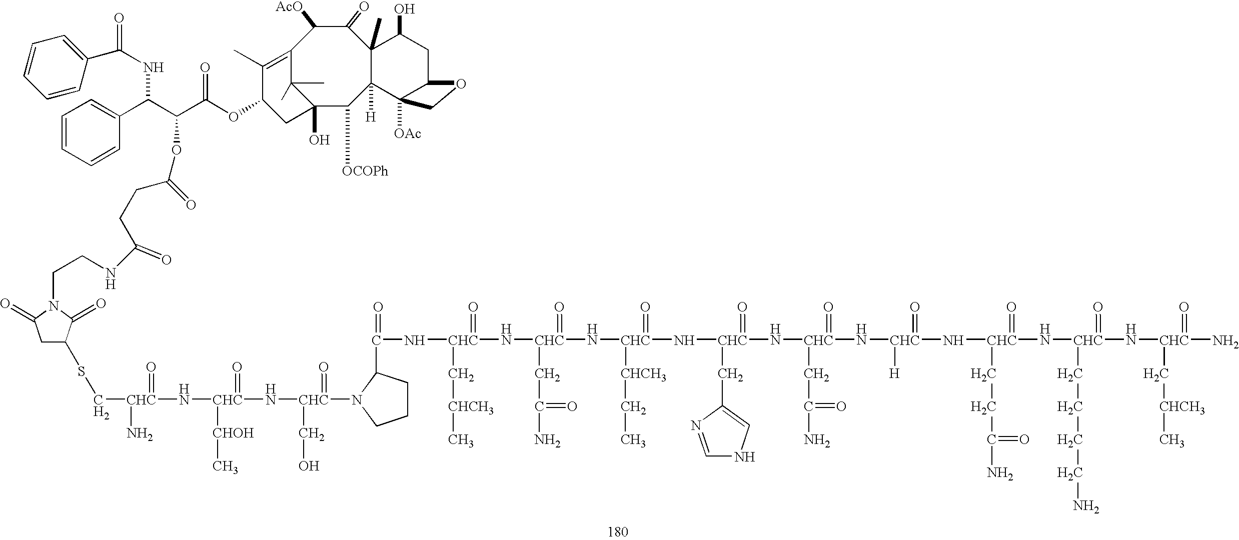

Synthesis of 2′-(4-Cbz-Aminobutyryl) Paclitaxel:

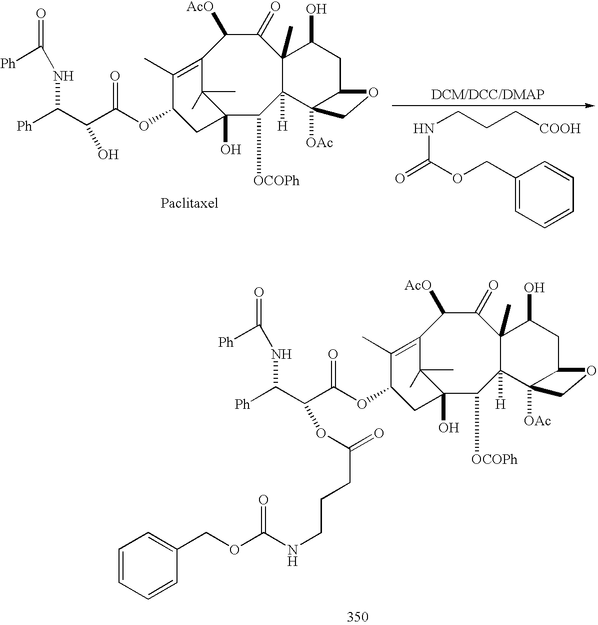

Paclitaxel 5.0 gms (5.86 mmols) was dissolved in 50 ml of DCM, N-CBZ-gamma-Amino-N-Butyric Acid 1.39 gms (5.86 mmols) was added under nitrogen with stirring followed DCC 1.21 gms (5.86 mmols) and finally DMAP 0.071 gms (0.586 mmols). The reaction was allowed to stir overnight at room temperature. TLC with 60% EtOAc/Heptane after 15 hours indicated the starting material had been converted to product (a faster spot). The solution was diluted with 50 ml DCM washed with 100 ml saturated bicarbonate, 100 ml water, dried over Na₂SO₄ and concentrated. The DCU was precipitated with 50 ml of MTBE, filtration of the precipitate and concentration of the filtrate yielded crude product. The crude was purified on a silica gel column with 60% EtOAc/Heptane to give 4.3 gms of pure product.

### Synthesis of 2′-Gamma-Aminobutyryl- Paclitaxel:

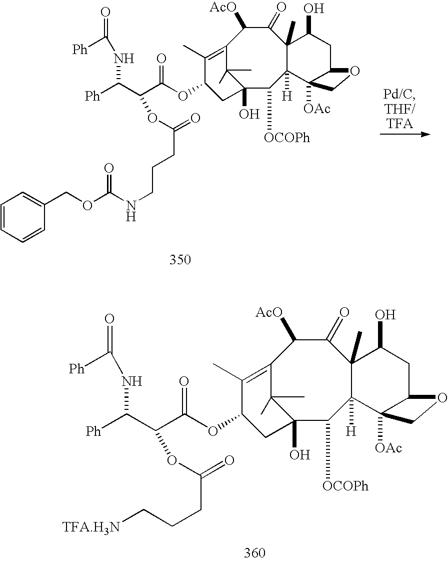

2′-(4-Cbz-aminobutyryl) paclitaxel 1.5 gms(1.4 mmols) was dissolved in 20 ml of THF. TFA 0.175 ml (1.53 mmols) was added followed by 0.1 gm 10% Pd/C. The 100 ml flask was purged with hydrogen and stirred under a double balloon of hydrogen for about 20 hours. The catalyst was filtered on celite and THF evaporated. The solid foam (TFA salt) was stored in the freezer and used as such in the next step.

### Synthesis of 2′-(Cis-Aconityl-Gamma-Aminobutyryl)Paclitaxel:

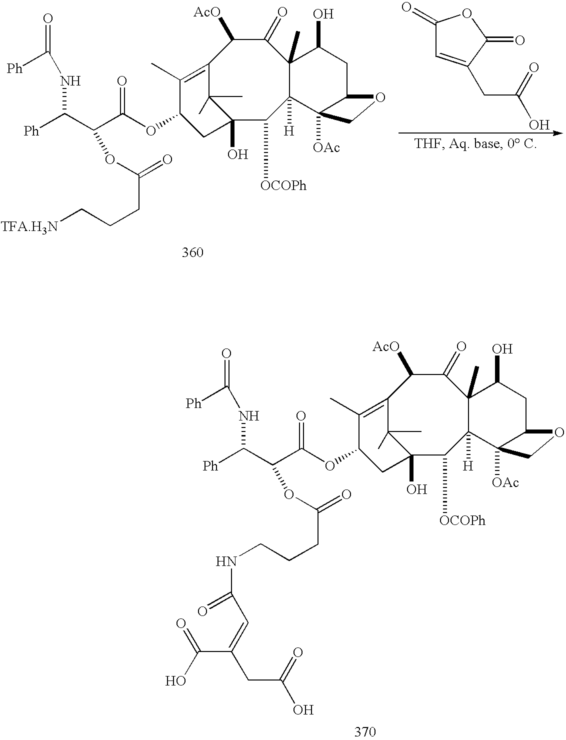

0.2 gms (0.19 mmols) of 2′-gamma-aminobutyryl-Paclitaxel TFA salt was dissolved in 20ml of THF and stirred as 10ml of water was added and the solution cooled on an ice bath. 5 ml of saturated bicarbonate was added followed by cis-aconitic anhydride 0.38 gms(2.4 mmols) in three portions. After 15 minutes the reaction was removed from the ice bath. Stirring was continued for about 55 minutes at room temperature after which the solution turned hazy. 10 ml each of THF and water were added followed by 200 mg of cis-aconitic anhydride. After stirring for another 10 minutes the THF was evaporated on the rotavapor at 25° C. The aqueous solution was acidified first with concentrated HCl and then IN HCl to pH 2.5-3.0 (pH paper was used to monitor pH). The precipitated foam was filtered and dried on vacuum to yield 160 mg of product which was used directly in couplings with Transferrin and peptides.

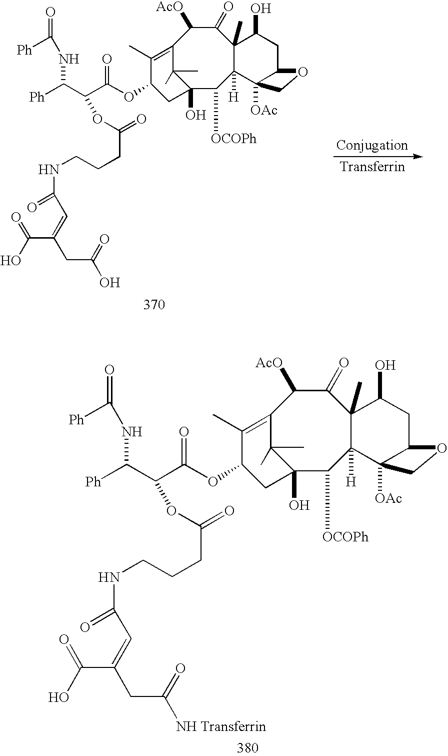

Other peptide and protein conjugates were prepared using the methods described above are as shown in the following Schemes:

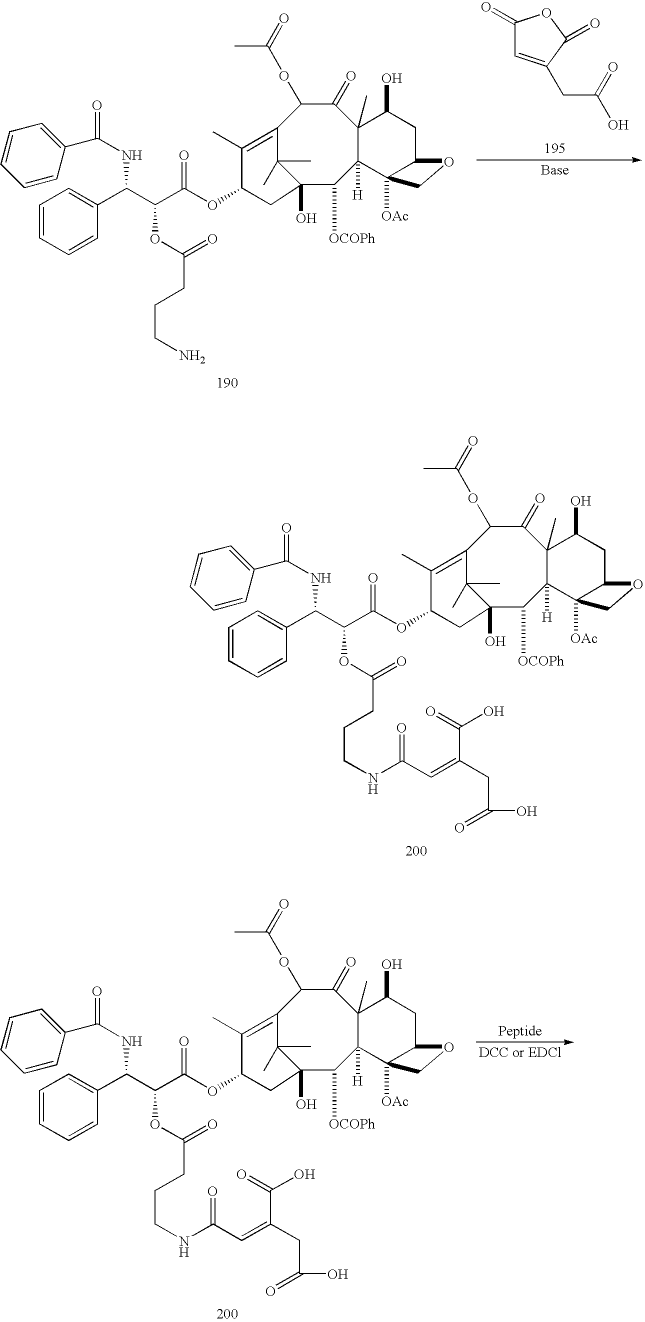

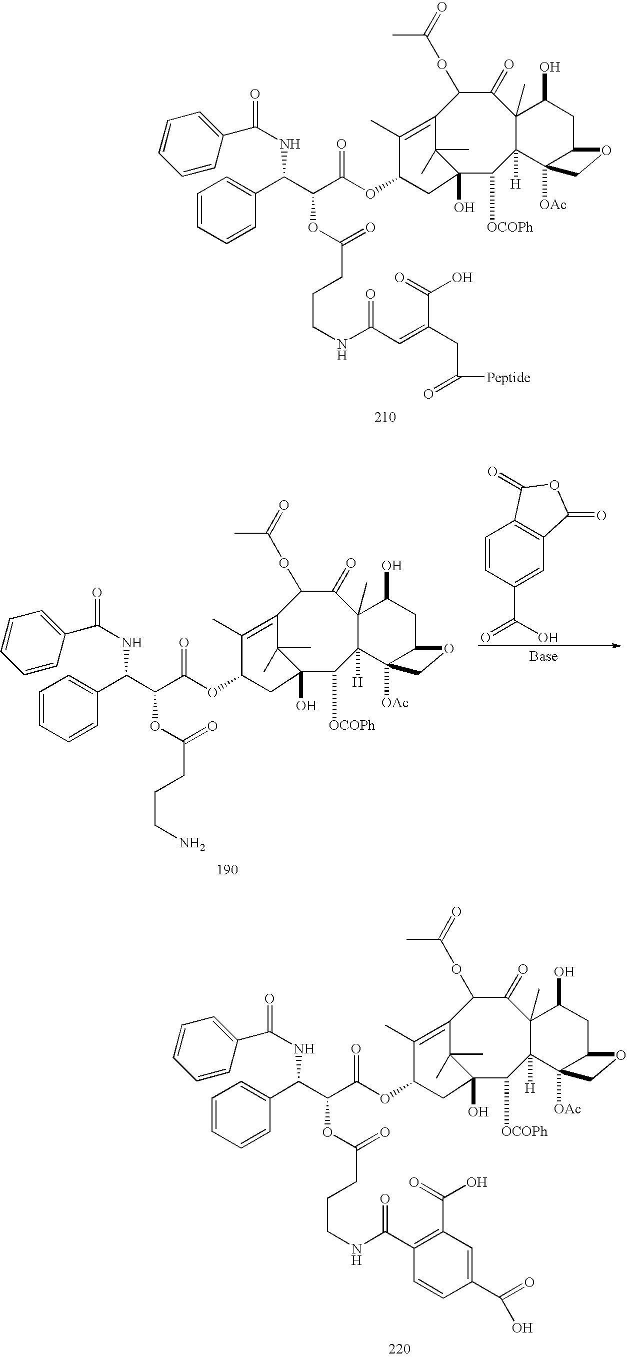

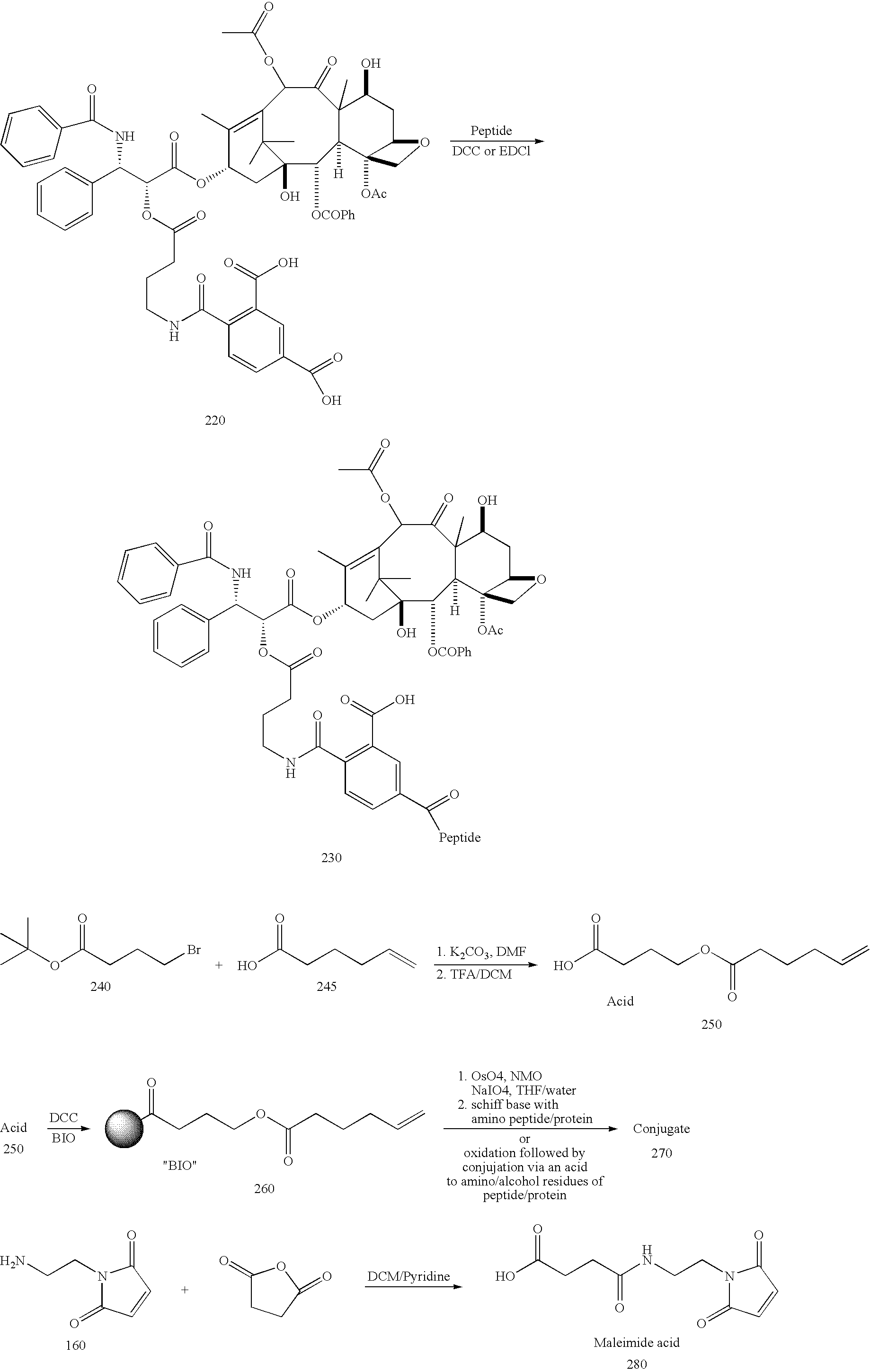

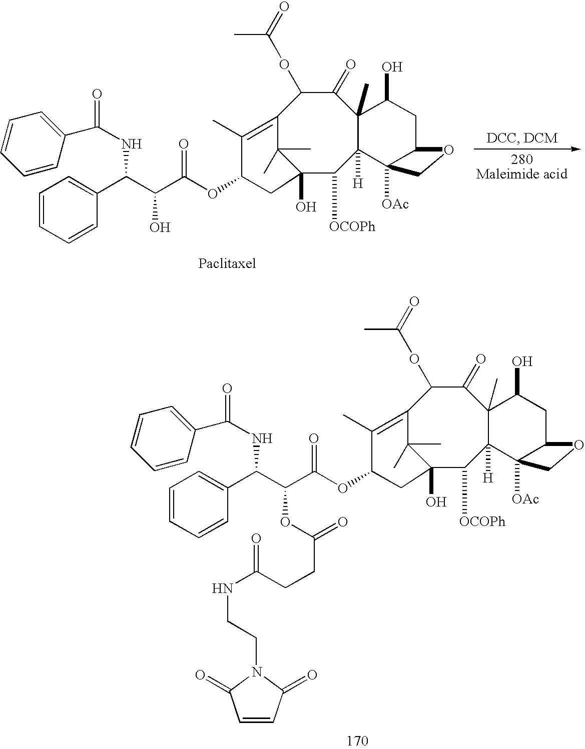

### Synthesis of Quassinoid Peptide Conjugates:

### Synthesis of 1-Glutaryl Quassinoid Compound 300:

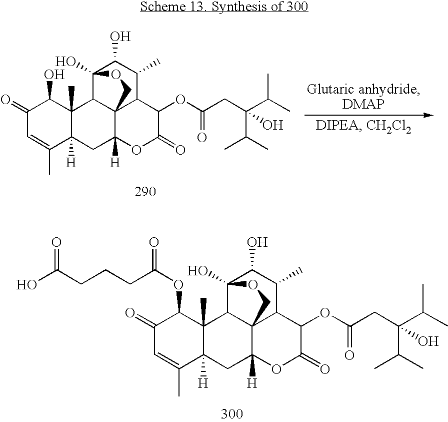

Added glutaric anhydride (0.061 g, 0.54 mmol), DMAP (0.012 g, 0.098 mmol) and DIPEA (0.18mL) sequentially to a solution of compound 290 (0.268 g, 0.488 mmol) in anhydrous CH₂Cl₂ at RT under N₂ atmosphere and stirred overnight. Confirmed completion of the reaction by TLC and the reaction mixture was diluted with CH₂Cl₂ (10 mL) and quenched with 10% citric acid solution (5 mL) at RT. The organic layer was separated, washed with water (10 mL), dried (Na₂SO₄) and concentrated in vacuo to afford the crude product which was then precipitated from acetone/water to afford clean product (0.245 g, 75.7%).

### Synthesis of 1-Glutaramidoethyl Maleimide Quassinoid 310:

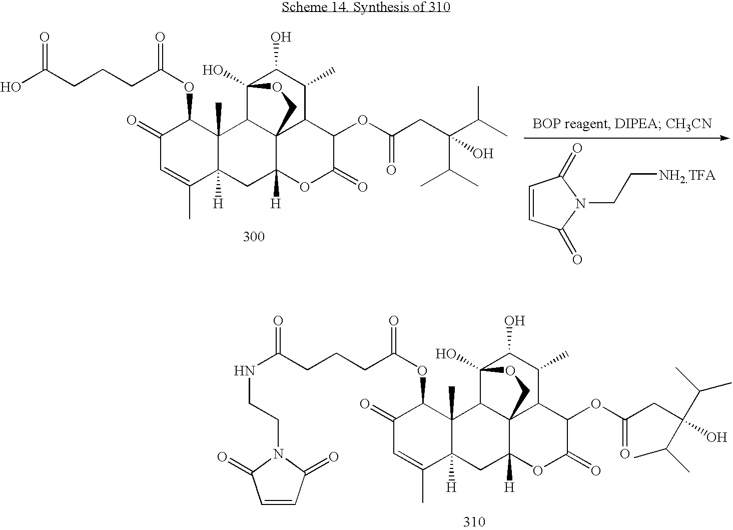

To a solution of the starting material compound 300 (0.116 g, 0.175 mmol) in anhydrous CH3CN (6 mL), at RT under N₂ atmosphere, was added BOP reagent (0.78 g, 0.175 mmol), DIPEA (0.06 mL, 0.35 mmol), and N-(2—aminoethyl)maleimide TFA salt (0.45 g, 0.175 mmol) and stirred overnight. TLC analysis revealed completion of the reaction. The reaction mixture was diluted with IPAC (30 mL) and washed with water (2×15 mL), brine (1×10 mL), dried (Na₂SO₄) rotostripped and purified on a silica plug (eluting with 15% methanol in CH₂Cl₂) to afford the clean product (0.127 g).

### Synthesis of Peptide Quassinoid Conjugate:

To a solution of 310 (0.083 g, 0.105 mmol) in THF (4 mL) was added a solution of the Cys-HN-1 peptide (0.210 g, 0.147 mmol) at RT and stirred overnight. LC-MS analysis (CM2-00X method i.e. method-2) revealed completion of the reaction. The reaction mixture was evaporated to dryness on the rotavapor and the residue was dissolved in 5% acetonitrile in water with sonication. The solution was loaded onto an SPE cartridge (C-18) and purified by a gradient solvent system using water and acetonitrile to afford the cleaned up conjugate.

The conjugates prepared according to the present invention can be readily processed by lyophilization to form stable powders and may be processed to form stable APIs.

The conjugates of the present invention are found to be more potent and also provide improved toxicity profile than the unconjugated biologically active compounds, such as the taxanes, their derivatives and analogs. Without being bound by any theory proposed herein, it is believed that the conjugates have increased potency because the conjugates penetrate the cell membranes more readily than the unconjugated analogs and are also less rapidly metabolized, and that the biologically active compounds are hydrolyzed in vivo to release the active compounds.

### Materials and Methods:

### FaDu Human Head and Neck Tumor Xenograft Model

Model Information—Female nude mice (nu/nu) between 5 and 6 weeks of age weighing approximately 20 g were obtained from Harlan, Inc. (Madison, Wis.). FaDu, obtained from the American Type Culture Collection (ATCC), is a head and neck tumor cell line originating from a punch biopsy of a hypopharyngeal tumor from a 56 year old Caucasian male. Animals were implanted subcutaneously (SC) by trocar with fragments of FaDu harvested from SC growing tumors in nude mice hosts. When tumors grew to approximately 84 mg in size (12 days following implantation), animals were pair-matched by tumor size into treatment and control groups; each group contained 8 mice. Animals were ear-tagged and followed individually throughout the experiment.

Dosing- Initial doses were given on Day 1 following pair matching. compound 180 in vehicle (sterile water) was administered IV at 62, 125, and 250 mg/kg on Days 1, 8, 15, and 22. Paclitaxel was administered by IV injection at 20 mg/kg (Days 1, 8, 15, and 22), or by intraperitoneal (IP) injection at 15 mg/kg (Days 1—5). Cisplatin (American Pharmaceutical Partners) was administered (IP) at 4 mg/kg on Days 1—5. To serve as negative controls, the compd 180 vehicle of sterile water was administered IV on Days 1,8,15, and 22, and a vehicle of 12.5% Cremophor EL, 12.5% EtOH and 75% Saline was administered IP on Days 1-5.

Antitumor Activity of Compound 180 conjugate in vivo: The antitumor effect of Compound 180 versus Taxol® was demonstrated in FaDu xenografts by administration every other day (qod), of Taxol® at 24 mg/kg or Compound 180 at 82.8 and 124 mg/kg. 2×106 FaDu cells were injected s.c. into the flank of nude mice. After the formation of palpable and measurable tumor nodules (approximately 10 days post implantation), mice were randomly assigned to treatment groups of ten mice each. Treatment was initiated on day 0 when the tumor volume reached approximately 20 mm . Taxol® and Compound 180 were injected intravenously at the doses indicated above. Control groups received 100 μl saline by i.v. administration. Tumor size and body weight were measured and monitored twice a week starting on day 0. Tumor volumes were measured two time a week, and body weight, tumor volume and survival curve were calculated and plotted by Prism 4.0 software as shown in FIGS. III a, b and c respectively.

Release of paclitaxel (TX) from compound 180 was studied in vitro in mouse plasma and human serum by analyzing known concentration of samples at regular intervals by LC/UV/MS. These results are plotted here (FIGS. IVa and IVb)

### Combination Therapy:

In accordance with the present invention, administration of the two components, the conjugate compounds of the present invention and a second chemotherapeutic agent, concomitantly or sequentially, synergistically enhances the treatment of cancer as compared to administering each component independently in monotherapy. The synergistic effect results in an improved therapeutic index as compared to either agent alone while toxicity remains acceptable. Preferably, the conjugate compound is administered to the patient in an oral unit dosage form, more preferably in capsule or tablet form. The second chemotherapeutic agent is administered by parenteral, preferably by intravenous administration, in association with a conjugate compound of the present invention, as described herein. The first chemotherapeutic agent and the second conjugate compound of the present invention are administered in any amount and for any duration that is effective to maintain or decrease tumor size.

The conjugates of the present invention are evaluated in combination with a second chemotherapeutic agent in vitro using a tetrazolium dye assay in seven different tumor cell lines derived from a variety of cancers. The results demonstrate that in cell culture studies with MDA-MB-435 (breast), H460a (lung), MES-SA/D×5 (uterine), LS513 (colon), MTLn3 (breast), LS1034 (colon), and MDA-MB-231 (breast) tumor cells, the conjugates of the present invention in combination with a second chemotherapeutic agent produce a statistically significant greater growth inhibitory effect than that produced by either compound alone at the same concentrations.

The results are most dramatic in MDA-MB-435 cells, where doses that provides 10—15% growth inhibition as single agents gave greater than 80% inhibition when combined. The in vitro studies demonstrate dose combinations of the conjugates with a second chemotherapeutic agent that provide superior antiproliferative activity compared to corresponding doses of these same agents in monotherapy.

Various modifications and variations of the above disclosed compounds may be prepared based on the teaching of the present disclosure without departing from the spirit or scope of the invention. The present invention is intended to cover such modifications and variations of this invention provided they come within the scope of the appended claims and their equivalents.

What is claimed is:

1. A compound comprising the formula I:
B—X—L¹—(L²)ₘ—(L³)ₙ—Y—RL I
wherein:
B is a biologically active agent, analogs or derivatives thereof;
L¹ is a linker comprising at least 2 atoms in the linker chain, wherein the atoms are selected from the group consisting of C, N, O and S;
L² is a linker comprising at least 2 atoms in the linker chain, wherein the atoms are selected from the group consisting of C, N, O and S;
L³ is a linker comprising at least 2 atoms in the linker chain, wherein the atoms are selected from the group consisting of C and N;
X and Y are each independently O, NR¹ or S;
RL is a ligand;
R¹ is hydrogen or a substituted or unsubstituted C₁₋₄alkyl or C₁₋₄alkylCO—;
m is 1, 2, 3, 4 or5;and n is 0, 1 or 2;or a pharmaceutically acceptable salt or prodrug thereof.

2. A compound comprising the formula II: 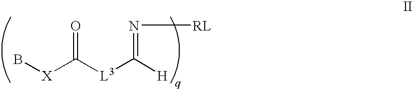
wherein:
B is a biologically active agent, analogs or derivatives thereof;
RL is a ligand;
R¹ is hydrogen or a substituted or unsubstituted C₁₋₄alkyl or C₁₋₄alkylCO—;
L³ is selected from the group consisting of (1) a C₁₋₂₀alkyl optionally substituted with one or more phenyl group, the C₁₋₂₀alkyl is optionally interrupted by 1 or 2 heteroatoms selected from the group consisting of O, N or S; (2) a C₃₋₂₀cycloalkyl group optionally substituted with one or more C₁₋₂₀alkyl or phenyl group; and (3) an aromatic group optionally substituted with one or more C₁₋₈alkyl or an electron-withdrawing or electron-donating groups;
X is O, NR¹ or S;
q is 1-5; or
a pharmaceutically acceptable salt or prodrug thereof.

3. The compound of claim 1 or 2, wherein the biologically active agent is selected from the group consisting of taxanes, camptothecins, epothilones, cucurbitacins, quassinoids, anthracyclines, and their analogs and derivatives.

4. The compound of claim 1, wherein the biologically active agent is a drug useful for cancer therapy.

5. A compound comprising the formula III:
TQ—O—L¹—(L²)ₘ—(L³)ₙ—Y—RL III
wherein:
TQ is a taxane or a quassinoid derivative;
L¹ is a linker comprising at least 2 atoms in the linker chain, wherein the atoms are selected from the group consisting of C, N, O and S;
L² is a linker comprising at least 2 atoms in the linker chain, wherein the atoms are selected from the group consisting of C, N, O and S;
L³ is a linker comprising at least 2 atoms in the linker chain, wherein the atoms are selected from the group consisting of C, N, O and S;
Y is O, NR¹ or S;
RL is a ligand;
R¹ is hydrogen or substituted or unsubstituted C₁₋₄alkyl;
m is 1, 2, 3, 4 or 5; and n is 0, 1 or 2; or a pharmaceutically acceptable salt or prodrug thereof.

6. The compound of claim 5 wherein the taxane derivative is selected from the group consisting of a paclitaxel, 7-dehydroxyl paclitaxel or docetaxel, 10-dehydroxyl paclitaxel, docetaxel or compound 70, 2′-dehydroxyl paclitaxel, 3′-de-benzamido paclitaxel and their analogs, prodrug and derivatives thereof.

7. (canceled)

8. The compound of claim 5 wherein the taxane derivative is attached to linker L¹ at the 2′-, 1-, 7—or 10-hydroxyl position of the taxane moiety.

9.-10. (canceled)

11. The compound of claim 5 wherein the ligand is selected from the group consisting of an HN-1 peptide, bombesin/gastrin-releasing peptide (BBN/GRP) receptor-recognizing peptide (BBN[7-13]), a somatostatin receptor recognizing peptide, an LHRH receptor recognizing peptide, an epidermal growth factor receptor recognizing peptide, a monoclonal antibody, a receptor recognizing carbohydrate, a receptor ligand peptide, an endogenous peptide that targets the tumors with or without internalization, carrier molecules including peptides, proteins, Transferrin, an antibody, lextins and agents that attaches to the surface of a cell.

12. (canceled)

13. The compound of claim 5, wherein L¹ is a substituted or unsubstituted dicarbonyl compound selected from the group consisting of 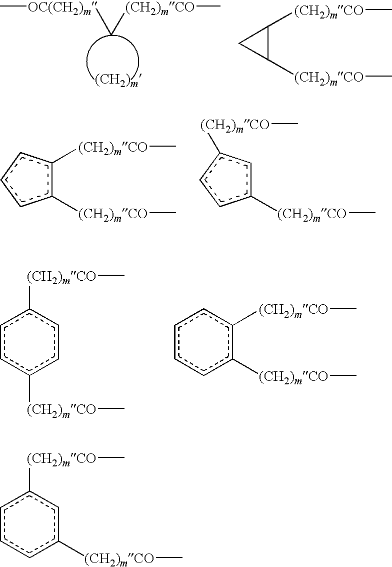
wherein m′ is 2-7; and each m″ is independently 0, 1, 2, 3, 4 or 5.

14. The compound of claim 5, wherein L¹ is selected from the group consisting of substituted or unsubstituted —C(O)(CH₂)₂₋₅C(O)—, —C(O)CH₂CH(CH₃)CH₂C(O)—, —C(O)CH₂C(CH₃)₂CH₂C(O)—, —C(O)CH₂CH(CH₂CH₃)CH₂C(O)—, —C(O)CH₂C(CH₂CH₃)₂CH₂C(O)—, —C(O)(CH₂)₂₋₅CH(CH₃)CH₂C(O)—, —C(O)(CH₂)₂₋₅C(CH₃)₂CH₂C(O)—, —C(O)(CH₂)₂₋₅CH(CH₂CH₃)CH₂C(O)—, —C(O)(CH₂)₂₋₅C(CH₂CH₃)₂CH₂C(O)—, —S(O)(CH₂)₂₋₅S(O)—, —S(O)CH₂CH(CH₃)CH₂S(O)—, —S(O)CH₂C(CH₃)₂CH₂S(O)—, —S(O)CH₂CH(CH₂CH₃)CH₂S(O)—, —S(O)CH₂C(CH₂CH₃)₂CH₂S(O)—, —S(O)(CH₂)₂₋₅CH(CH₃)CH₂S(O)—, —S(O)(CH₂)₂₋₅C(CH₃)₂CH₂S(O)—, —S(O)(CH₂)₂₋₅CH(CH₂CH₃)CH₂S(O)—, —S(O)(CH₂)₂₋₅C(CH₂CH₃)₂CH₂S(O)—, —C(O)(CH₂)₂₋₅NHC(O)—, —C(O)NH(CH₂)₂₋₅OC(O)—, —C(O)(CH₂)₂₋₅OC(O)—, —C(O)O(CH₂)₂₋₅OC(O)—, —C(O)(CH₂)₂₋₅SC(O)—, —C(O)S(CH₂)₂₋₅OC(O)—, —S(O)(CH₂)₂₋₅NHS(O)—, —S(O)NH(CH₂)₂₋₅S(O)—, —S(O)(CH₂)₂₋₅OS(O)— and —S(O)O(CH₂)₂₋₅S(O)—.

15-17. (canceled)

18. The compound of claims 13 or 14, wherein the substituent is in the position that is alpha to one of the carbonyl, sulfonyl, sulfonamide or sulfone groups.

19. The compound of claim 18, wherein the substituent on the dicarbonyl, disulfonyl, sulfone or sulfonamide compound comprises 1, 2 or 3 substituents selected from the group consisting of halo, hydroxy, cyano, aryloxy, silyloxy, C₁₋₄alkyl, C₁₋₄alkoxy, C₁₋₈alkylamino, C₆₋₁₀aryl and C₄₋₁₂heteroaryl.

20. The compound of claim 5, wherein L² is selected from the group consisting of substituted or unsubstituted —(CH₂)₁₋₅—, —NH(CH₂)₁₋₅—, —NH(CH₂)₂₋₅O—, —O(CH₂CH₂O)₀₋₂₀(CH₂)₂O—, —NH(CH₂)₂(OCH₂CH₂)₃NH—, —NH(CH₂CH₂O)₃(CH₂)₂NHC(O)—, —NH(CH₂CH₂O)₂CH₂CH₂NH—, —NHCH₂CH₂(OCH₂CH₂)₂NHC(O)—, —NH(CH₂)₂O(CH₂)₂NH—, —NH(CH₂)₂O(CH₂)₂NHC(O)—, —O(CH₂CH₂O)₃(CH₂)₂NH—, —O(CH₂CH₂O)₃(CH₂)₂NHC(O)—, —O(CH₂CH₂O)₂(CH₂)₂NH—, —O(CH₂CH₂O)₂(CH₂)₂NHC(O)—, —OCH₂CH₂O(CH₂)₂NH—, —O(CH₂)₂O(CH₂)₂NHC(O)—, —OCH(CH₃)C(O)—, —O(CH₂)₂₋₆—, —O(CH₂)₂₋₆NH—, —OCH₂CH₂O—, —O(CH₂)₃₋₆O—, —NH(CH₂)₂₋₆—, —NH(CH₂)₂₋₆NH—and —NH(CH₂)₂₋₆O—.

21. (canceled)

22. The compound of claim 5, wherein L² is —C(O)CH(OR²)CH(OR³)C(O)—, wherein R² and R³ are each independently hydrogen or C₁₋₄alkyl.

23. The compound of claim 5, wherein L³ is a substituted or unsubstituted moiety selected from the group consisting of

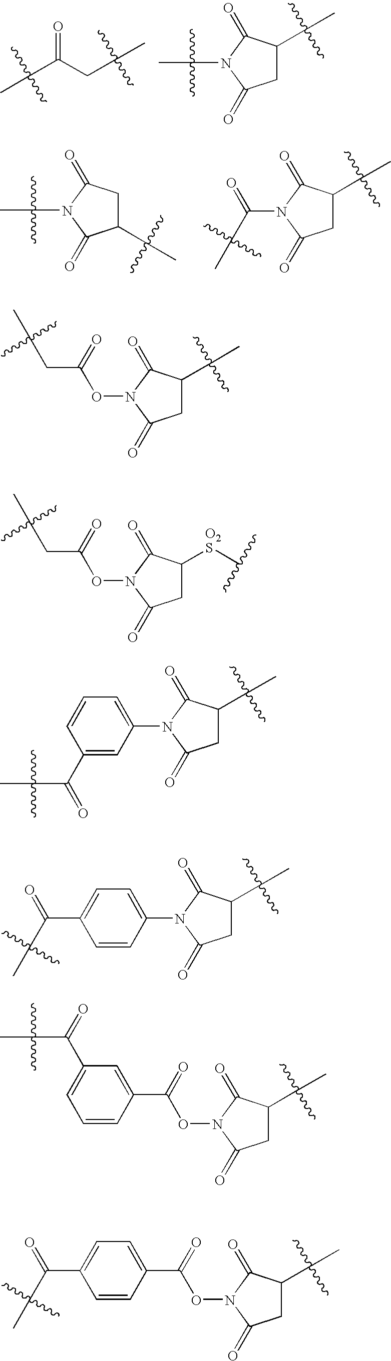

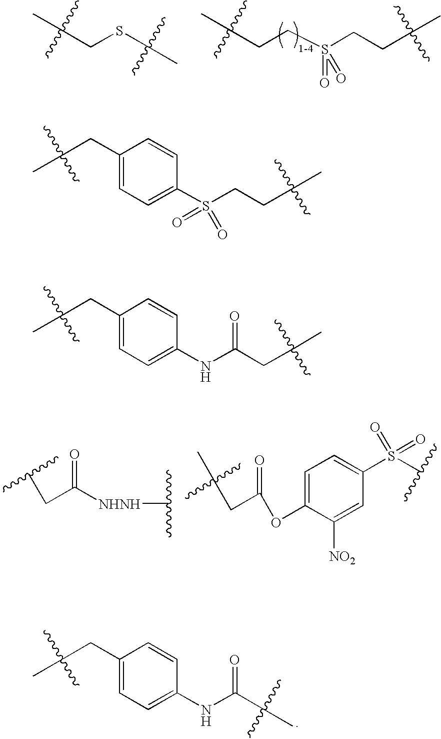

24-25. (canceled)

26. The compound of claim 5, wherein the group represented by —O—L¹—(L²)ₘ—(L³)ₙ— is a moiety selected from the group consisting of substituted or unsubstituted 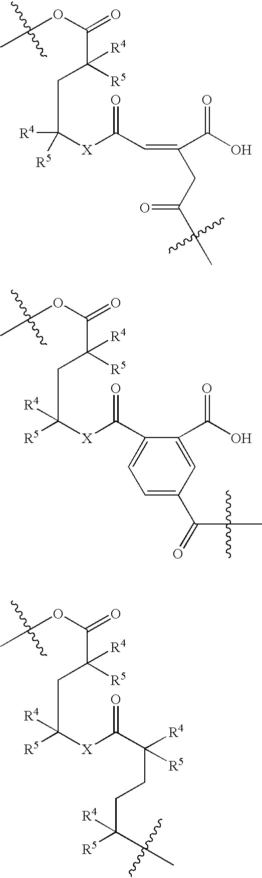
wherein:
X is selected from the group consisting of O, NR°, and S;
R° is hydrogen or substituted or unsubstituted C₁₋₄alkyl and C₁₋₄alkylCO—; and
each R⁴ and R⁵ is independently selected from the group consisting of hydrogen, hydroxy, C₁₋₄alkyl, C₁₋₄alkoxy and halo, or wherein R⁴ and R⁵ together are oxo.

27. The compound of claim 26, wherein each substituent independently comprises 1, 2 or 3 substituents independently selected from the group consisting of halo, hydroxy, cyano, C₁₋₄alkyl, C₁₋₄alkoxy, C₁₋₈alkylamino, C₆₋₁₀aryl and C₄₋₁₂heteroaryl.

28. The compound of claim 27 wherein Y is S.

29. The compound of claim 5, wherein RL—Y— is the N-terminal cysteine sulfhydryl group of cysteine bearing HN-1.

30. A compound comprising the formula: 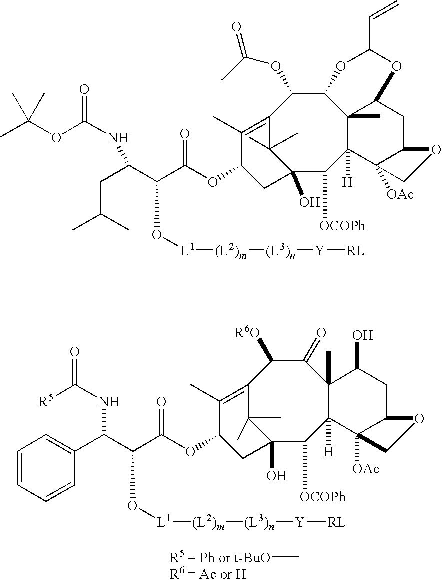
wherein
L¹ is a linker comprising at least 2 atoms in the linker chain, wherein the atoms are selected from the group consisting of C, N, O and S;
L² is a linker comprising at least 2 atoms in the linker chain, wherein the atoms are selected from the group consisting of C, N, O and S;
L³ is a linker comprising at least 2 atoms in the linker chain, wherein the atoms are selected from the group consisting of C and N;
Y is O, NR¹ or S;
RL is a ligand;
R¹ is hydrogen or a substituted or unsubstituted C₁₋₄alkyl or C₁₋₄alkylCO—;
m is 1, 2, 3, 4 or 5; and n is 0, 1 or 2; or
a pharmaceutically acceptable salt or prodrug thereof.

31. A compound comprising the formulae: 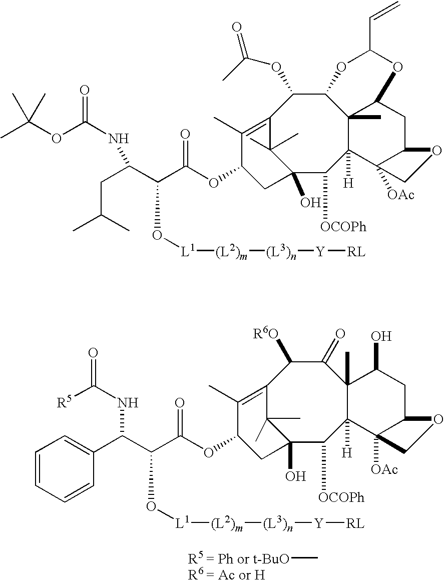
wherein:
L¹ is a linker selected from the group consisting of a substituted or unsubstituted dicarbonyl compound; 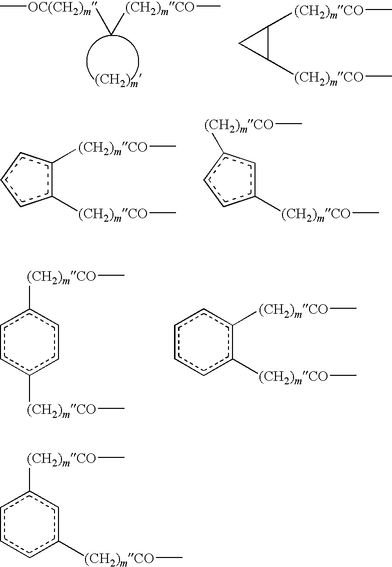
wherein m′ is 2-7; and each m″ is independently 0, 1, 2, 3, 4 or 5;
substituted or unsubstituted —C(O)(CH₂)₂₋₅C(O)—, —C(O)CH₂CH(CH₃)CH₂C(O)—, —C(O)CH₂C(CH₃)₂CH₂C(O)—, —C(O)CH₂CH(CH₂CH₃)CH₂C(O)—, —C(O)CH₂C(CH₂CH₃)₂CH₂C(O)—, —C(O)(CH₂)₂₋₅CH(CH₃)CH₂C(O)—, —C(O)(CH₂)₂₋₅C(CH₃)₂CH₂C(O)—, —C(O)(CH₂)₂₋₅CH(CH₂CH₃)CH₂C(O)—, —C(O)(CH₂)₂₋₅C(CH₂CH₃)₂CH₂C(O)—; —S(O)(CH₂)₂₋₅S(O)—, —S(O)CH₂CH(CH₃)CH₂S(O)—, —S(O)CH₂C(CH₃)₂CH₂S(O)—, —S(O)CH₂CH(CH₂CH₃)CH₂S(O)—, —S(O)CH₂C(CH₂CH₃)₂CH₂S(O)—, —S(O)(CH₂)₂₋₅CH(CH₃)CH₂S(O)—, —S(O)(CH₂)₂₋₅C(CH₃)₂CH₂S(O)—, —S(O)(CH₂)₂₋₅CH(CH₂CH₃)CH₂S(O)—, —S(O)(CH₂)₂₋₅C(CH₂CH₃)₂CH₂S(O)—; —C(O)(CH₂)₂₋₅NHC(O)—, —C(O)NH(CH₂)₂₋₅OC(O)—, —C(O)(CH₂)₂₋₅OC(O)—, —C(O)O(CH₂)₂₋₅OC(O)—, —C(O)(CH₂)₂₋₅SC(O)—, —C(O)S(CH₂)₂₋₅OC(O)—; —S(O)(CH₂)₂₋₅NHS(O)—, —S(O)NH(CH₂)₂₋₅S(O)—, —S(O)(CH₂)₂₋₅OS(O)—and —S(O)O(CH₂)₂₋₅S(O)—;
L² is a linker selected from the group consisting of a substituted or unsubstituted —(CH₂)₁₋₅—, —NH(CH₂)₁₋₅, —NH(CH₂)₂₋₅O—, —O(CH₂CH₂O)₀₋₂₀(CH₂)₂O—, —NH(CH₂)₂(OCH₂CH₂)₃NH—, —NH(CH₂CH₂O)₃(CH₂)₂NHC(O)—, —NH(CH₂CH₂O)₂CH₂CH₂NH—, —NHCH₂CH₂(OCH₂CH₂)₂NHC(O)—, —NH(CH₂)₂O(CH₂)₂NH—, —NH(CH₂)₂O(CH₂)₂NHC(O)—, —O(CH₂CH₂O)₃(CH₂)₂NH—, —O(CH₂CH₂O)₃(CH₂)₂NHC(O)—, —O(CH₂CH₂O)₂(CH₂)₂NH—, —O(CH₂CH₂O)₂(CH₂)₂NHC(O)—, —OCH₂CH₂O(CH₂)₂NH—, —O(CH₂)₂O(CH₂)₂NHC(O)—; —OCH(CH₃)C(O)—, —O(CH₂)₂₋₆—, —O(CH₂)₂₋₆NH—, —OCH₂CH₂O—, —O(CH₂)₃₋₆O—, —NH(CH₂)₂₋₆—, —NH(CH₂)₂₋₆NH—, —NH(CH₂)₂₋₆O—; and —C(O)CH(OR²)CH(OR³)C(O)—, wherein R² and R³ are each independently hydrogen or C₁₋₄alkyl;
L³ is a linker selected from the group consisting of a substituted or unsubstituted moiety that is 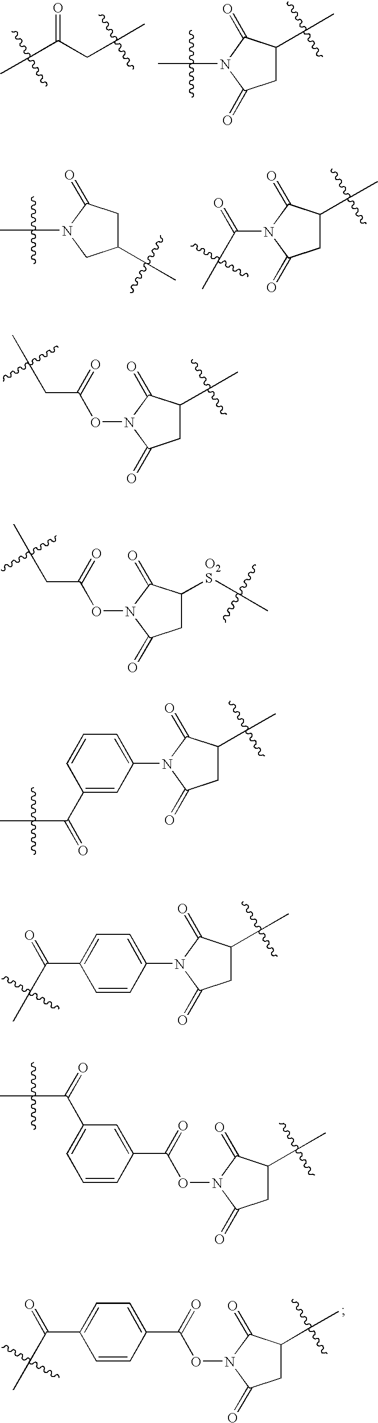 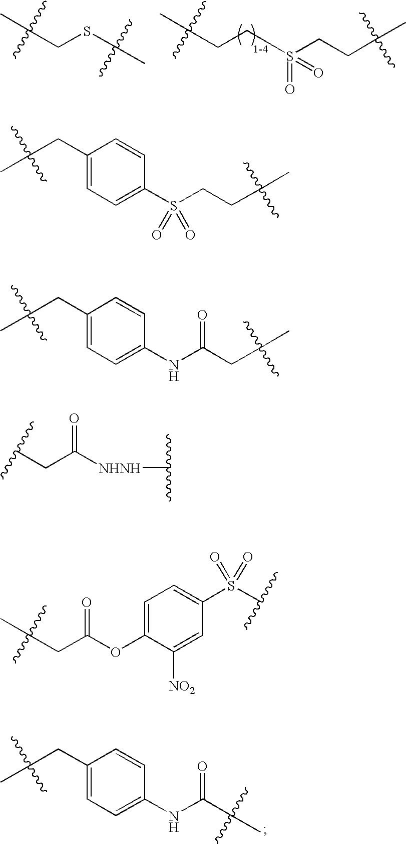
Y is O, NR¹ or S;
RL is a ligand; ^{R}¹ is hydrogen or a substituted or unsubstituted C₁₋₄alkyl or C₁₋₄alkylCO—;
m is 1, 2, 3, 4 or 5;and n is 0, 1 or 2;or
a pharmaceutically acceptable salt or prodrug thereof.

32. A compound selected from the group consisting of the formulae: 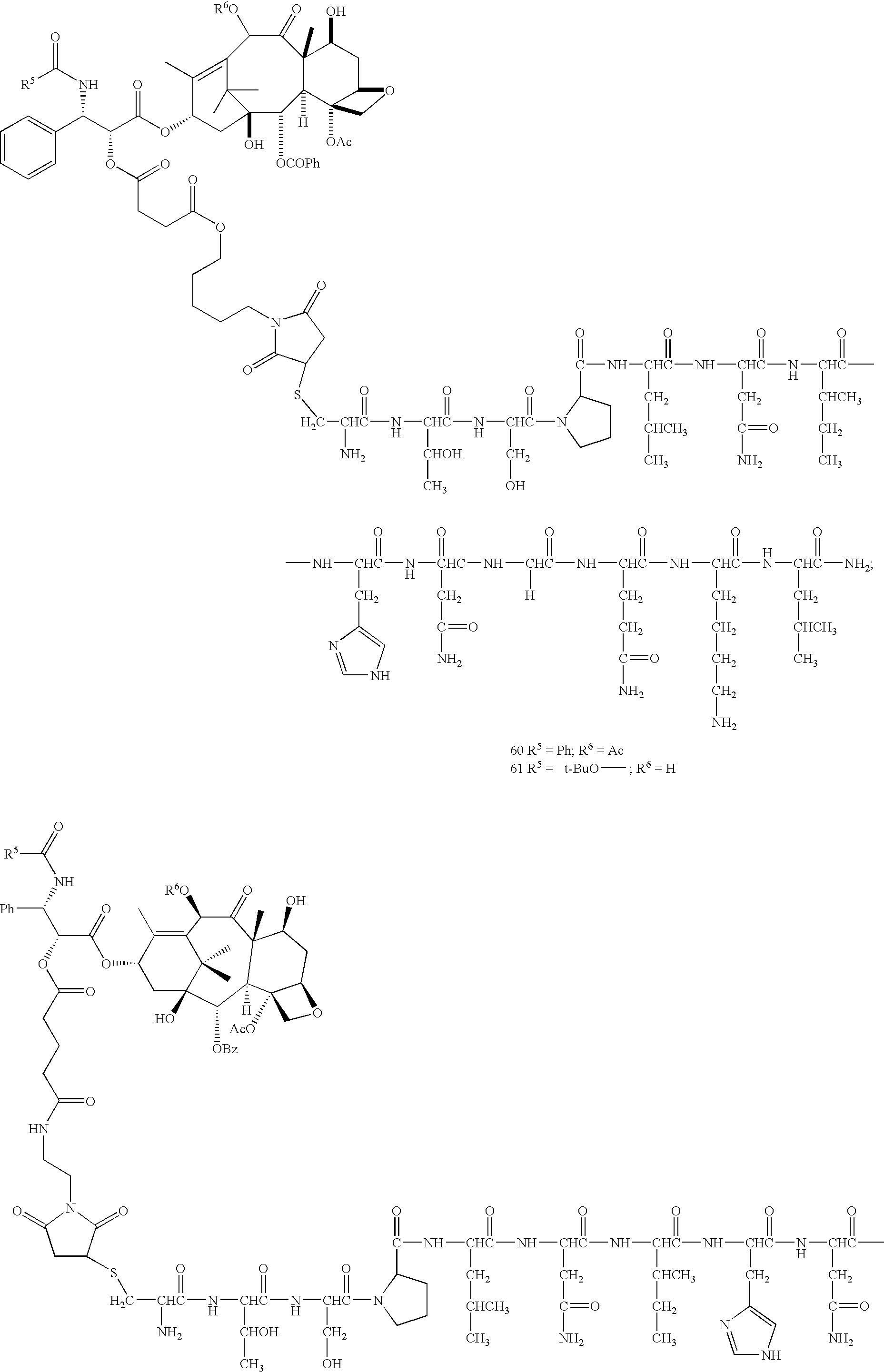 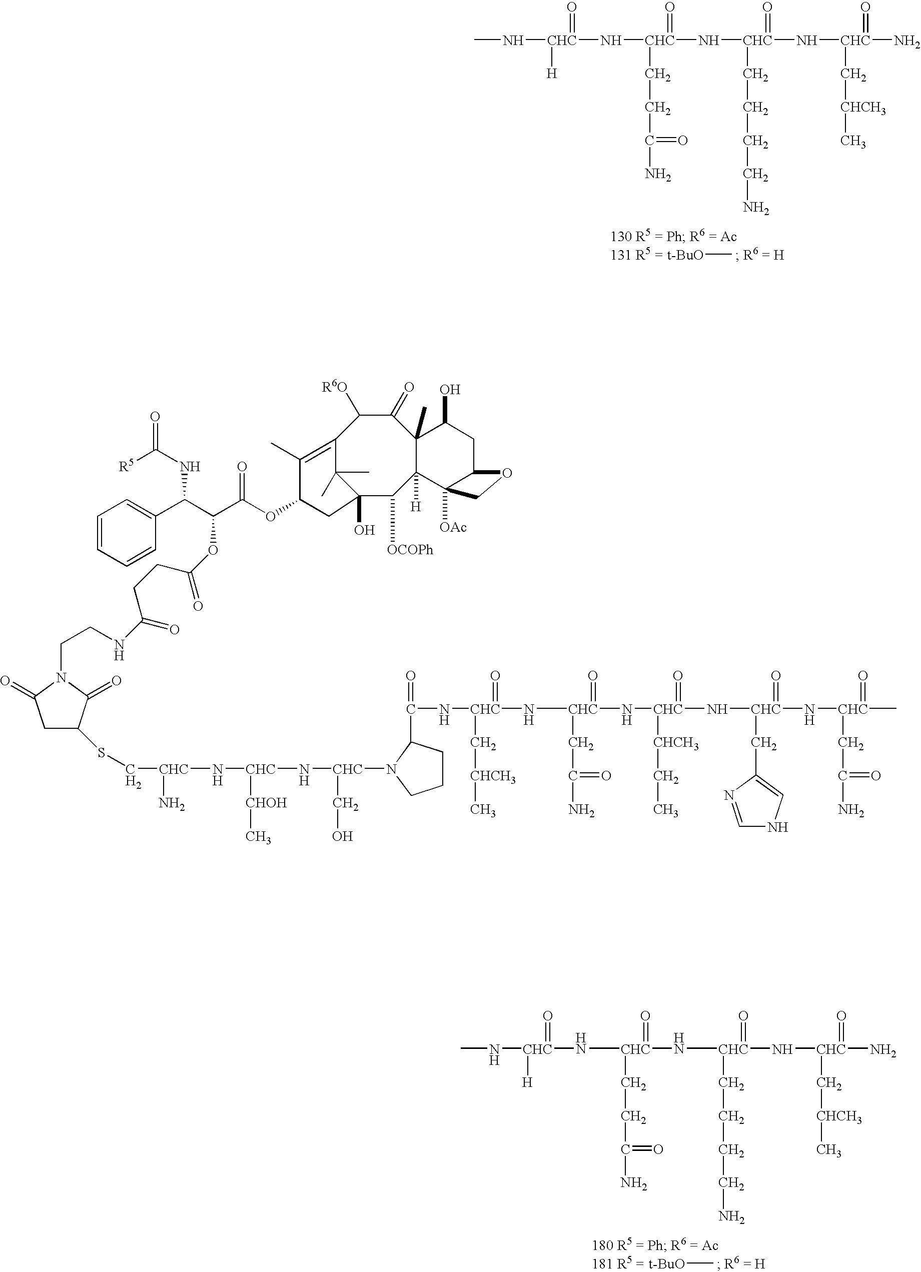 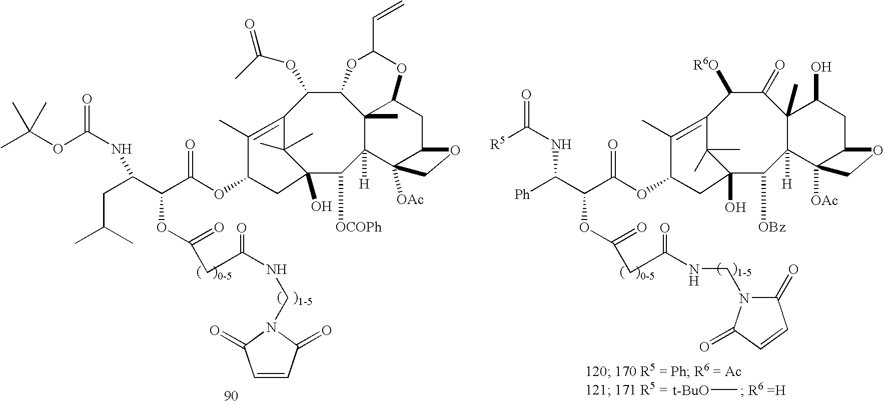
or a pharmaceutically acceptable salt thereof.

33. A compound comprising the formula: 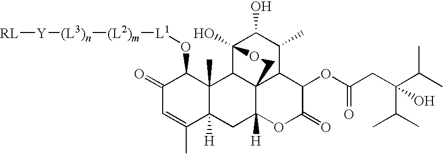
wherein:
L¹ is a linker comprising at least 2 atoms in the linker chain, wherein the atoms are selected from the group consisting of C, N, O and S;
L² is a linker comprising at least 2 atoms in the linker chain, wherein the atoms are selected from the group consisting of C, N, O and S;
L³ is a linker comprising at least 2 atoms in the linker chain, wherein the atoms are selected from the group consisting of C and N;
Y is O, NR¹ or S;
RL is a ligand;
R¹ is hydrogen or a substituted or unsubstituted C₁₋₄alkyl or C₁₋₄alkylCO—;
m is 1, 2, 3, 4 or 5; and n is 0, 1 or 2; or
a pharmaceutically acceptable salt or prodrug thereof.

34. A compound comprising the formula: 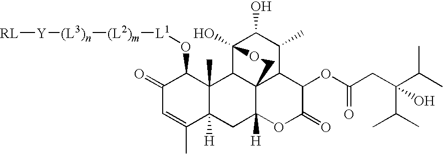
wherein:
L¹ is a linker selected from the group consisting of a substituted or unsubstituted dicarbonyl compound; 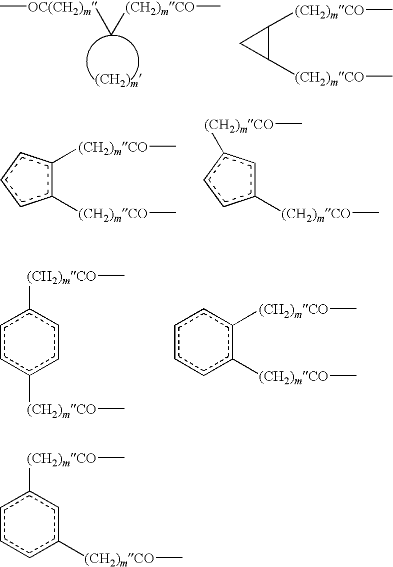
wherein m′ is 2-7; and each m″ is independently 0, 1, 2, 3, 4 or 5;
substituted or unsubstituted —C(O)(CH₂)₂₋₅C(O)—, —C(O)CH₂CH(CH₃)CH₂C(O)—, —C(O)CH₂C(CH₃)₂CH₂C(O)—, —C(O)CH₂CH(CH₂CH₃)CH₂C(O)—, —C(O)CH₂C(CH₂CH₃)₂CH₂C(O)—, —C(O)(CH₂)₂₋₅CH(CH₃)CH₂C(O)—, —C(O)(CH₂)₂₋₅C(CH3)₂CH₂C(O)—, —C(O)(CH₂)₂₋₅CH(CH₂CH₃)CH₂C(O)—, —C(O)(CH₂)₂₋₅C(CH₂CH₃)₂CH₂C(O)—; —S(O)(CH₂)₂₋₅S(O)—, —S(O)CH₂CH(CH₃)CH₂S(O)—, —S(O)CH₂C(CH₃)₂CH₂S(O)—, —S(O)CH₂CH(CH₂CH₃)CH₂S(O)—, —S(O)CH₂C(CH₂CH₃)₂CH₂S(O)—, —S(O)(CH₂)₂₋₅CH(CH₃)CH₂S(O)—, —S(O)(CH₂)₂₋₅C(CH₃)₂CH₂S(O)—, —S(O)(CH₂)₂₋₅CH(CH₂CH₃)CH₂S(O)—, —S(O)(CH₂)₂₋₅C(CH₂CH₃)₂CH₂S(O)—; —C(O)(CH₂)₂₋₅NHC(O)—, —C(O)NH(CH₂)₂₋₅OC(O)—, —C(O)(CH₂)₂₋₅OC(O)—, —C(O)O(CH₂)₂₋₅OC(O)—, —C(O)(CH₂)₂₋₅SC(O)—, —C(O)S(CH₂)₂₋₅OC(O)—; —S(O)(CH₂)₂₋₅NHS(O)—, —S(O)NH(CH₂)₂₋₅S(O)—, —S(O)(CH₂)₂₋₅OS(O)—and —S(O)O(CH₂)₂₋₅S(O)—;
L² is a linker selected from the group consisting of a substituted or unsubstituted group that is —(CH₂)₁₋₅—, —NH(CH₂)₁₋₅—, —NH(CH₂)₂₋₅O—, —O(CH₂CH₂O)₀₋₂₀(CH₂)₂O—, —NH(CH₂)₂(OCH₂CH₂)₃NH—, —NH(CH₂CH₂O)₃(CH₂)₂NHC(O)—, —NH(CH₂CH₂O)₂CH₂CH₂NH—, —NHCH₂CH₂(OCH₂CH₂)₂NHC(O)—, —NH(CH₂)₂O(CH₂)₂NH—, —NH(CH₂)₂O(CH₂)₂NHC(O)—, —O(CH₂CH₂O)₃(CH₂)₂NH—, —O(CH₂CH₂O)₃(CH₂)₂NHC(O)—, —O(CH₂CH₂O)₂(CH₂)₂NH—, —O(CH₂CH₂O)₂(CH₂)₂NHC(O)—, —OCH₂CH₂O(CH₂)₂NH—, —O(CH₂)₂O(CH₂)₂NHC(O)—; —OCH(CH₃)C(O)—, —O(CH₂)₂₋₆—, —O(CH₂)₂₋₆NH—, —OCH₂CH₂O—, —O(CH₂)₃₋₆O—, —NH(CH₂)₂₋₆—, —NH(CH₂)₂₋₆NH—, —NH(CH₂)₂₋₆O—; and —C(O)CH(OR²)CH(OR³)C(O)—, wherein R² and R³ are each independently hydrogen or C₁₋₄alkyl;
L³ is a linker selected from the group consisting of a substituted or unsubstituted moiety that is 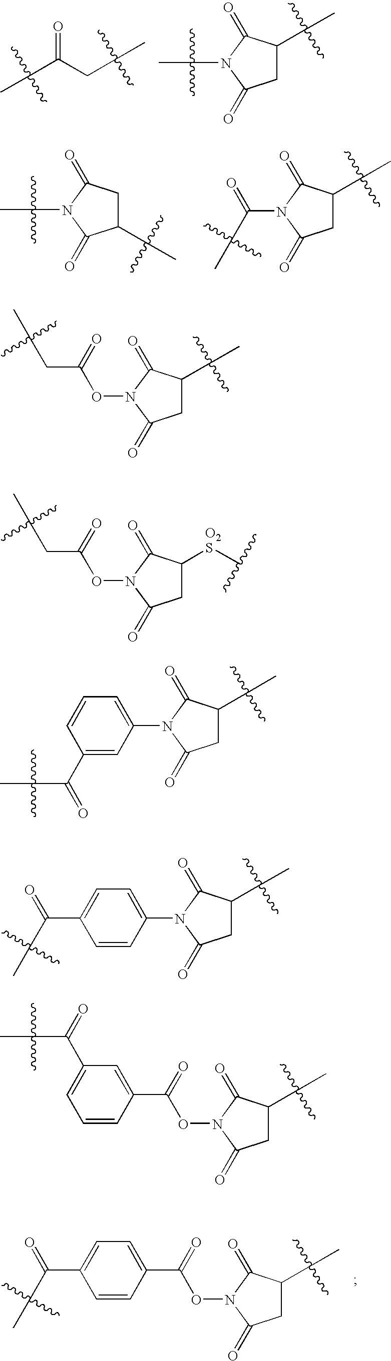 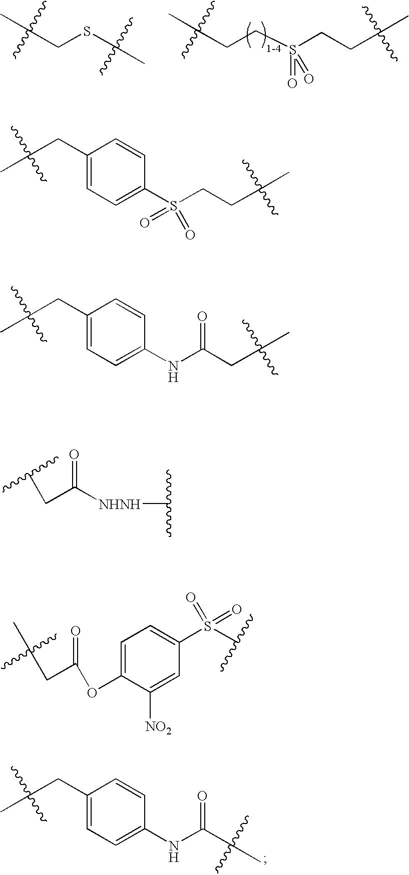
Y is O, NR¹ or S;
RL is a ligand;
R¹ is hydrogen or a substituted or unsubstituted C₁₋₄alkyl or C₁₋₄alkylCO—;
m is 1, 2, 3, 4 or 5; and n is 0, 1 or 2; or
a pharmaceutically acceptable salt or prodrug thereof.

35. The compound of any one of claims 30-34 wherein the ligand is selected from the group consisting of an HN-1 peptide, bombesin/gastrin-releasing peptide (BBN/GRP) receptor-recognizing peptide (BBN[7-13]), a somatostatin receptor recognizing peptide, an LHRH receptor recognizing peptide, an epidermal growth factor receptor recognizing peptide, a monoclonal antibody, a receptor recognizing carbohydrate, a receptor ligand peptide and an endogenous peptide that targets the tumors with or without internalization.

36. A compound selected from the group consisting of the formulae: 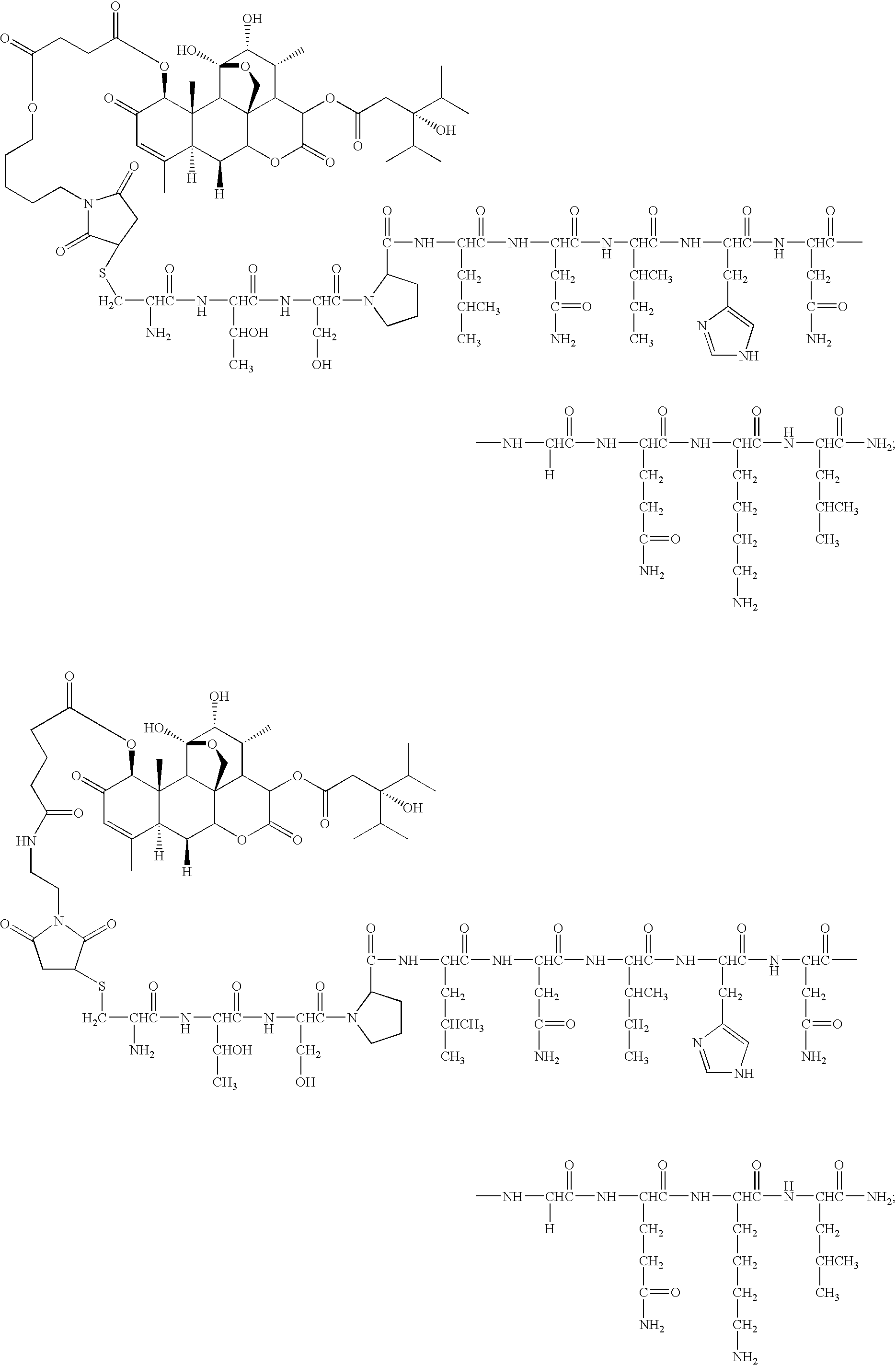 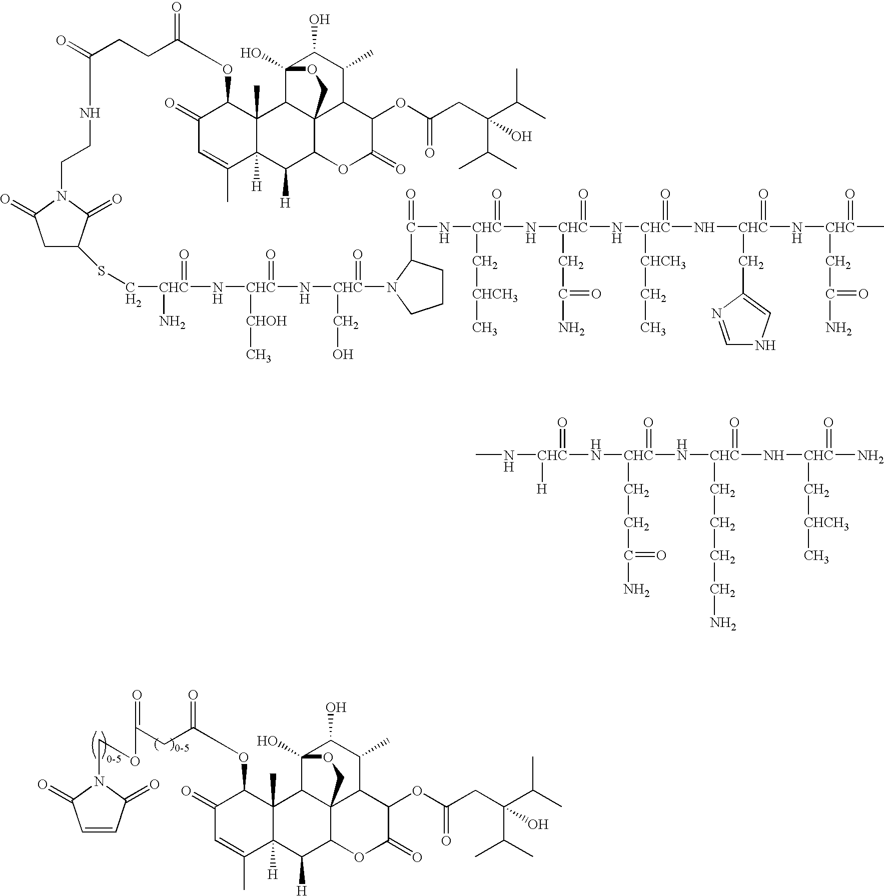
or a pharmaceutically acceptable salt thereof.

37. The compound of claim 1, wherein the group represented by —X—L¹—(L²)ₘ—(L³)ₙ— or the group represented by —X—L¹— is a substituted or unsubstituted moiety of the formula 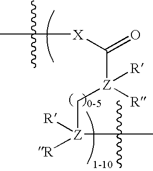
wherein:
each X is independently selected from the group consisting of O, S and NR¹;
each Z is independently selected from N, C, O, and S, provided that the structure does not form two adjacent Z groups that are both N, O or S;
R′ and R″ are each independently selected from the group consisting of H, hydroxy, halo, alkyl, alkyl, aryl, heteroaryl, alkoxy, and amino, each unsubstituted or substituted, or wherein R′ and R″ together are oxo; and
R¹ is hydrogen or a substituted or unsubstituted C₁₋₄alkyl or C₁₋₄alkylCO—.

38. A pharmaceutical composition comprising, as an active ingredient, a compound of claim 5.

39. A method for the treatment or prophylaxis of cancer or prevention of metastases from tumors comprising administration of a therapeutically effective amount of a composition of claim 38 to a patient in need of such a treatment that is used alone or in combination with radiotherapy and/or other chemotherapeutic treatments conventionally administered to patients for treating cancer.

40. (canceled)

41. A method for the treatment of cancer by the administration of an effective amount of a composition of claim 38, wherein the administration is performed with a chemotherapeutic agent selected from the group consisting of alpha interferon, COMP (cyclophosphamide, vincristine, methotrexate and prednisone), etoposide, mBACOD (methortrexate, bleomycin, doxorubicin, cyclophosphamide, vincristine and dexamethasone), PRO-MACE/MOPP (prednisone, methotrexate, doxorubicin; cyclophosphamide, taxol, etoposide/mechlorethamine, vincristine, prednisone and procarbazine), vincristine, vinblastine, angioinhibins, TNP—470, pentosan polysulfate, platelet factor 4, angiostatin, LM-609, SU-101, CM-101, Techgalan, thalidomide, alkylating agents, nitrogen mustards, mechloethamine, melphan, chlorambucil, cyclophosphamide and ifosfamide; nitrosoureas including carmustine, lomustine, semustine, streptozocin; alkyl sulfonates, busulfan; triazines, dacarbazine; ethyenimines, thiotepa, hexamethylmelamine; folic acid analogs, methotrexate; pyrimidine analogues, 5-fluorouracil, cytosine arabinoside; purine analogs, 6-mercaptopurine, 6-thioguanine; antitumor antibiotics, actinomycin D; anthracyclines, doxorubicin, bleomycin, mitomycin C, methramycin; hormones and hormone antagonists, tamoxifen, cortiosteroids; cisplatin and brequinar.

42. (canceled)

43. A method of treating a patient suffering with cancer comprising administering to the patient:
(i) a first component consisting of pharmaceutical composition comprising as an active ingredient a compound of formula I or formula III:
B—X—L¹—(L²)ₘ—(L³)ₙ—Y—RL I
TQ—O—L¹—(L²)ₘ—(L³)ₙ—Y—RL III
wherein:
B is a biologically active agent, analogs and derivatives thereof;
TQ is a taxane or a quassinoid derivative;
L¹ is a linker comprising at least 2 atoms in the linker chain, wherein the atoms are selected from the group consisting of C, N, O and S;
L² is a linker comprising at least 2 atoms in the linker chain, wherein the atoms are selected from the group consisting of C, N, O and S;
L³ is a linker comprising at least 2 atoms in the linker chain, wherein the atoms are selected from the group consisting of C and N;
X and Y are each independently O, NR¹ or S;
RL is a ligand;
R¹ is hydrogen or a substituted or unsubstituted C₁₋₄alkyl or C₁₋₄alkylCO—;
m is 1, 2, 3, 4 or 5; and n is 0, 1 or 2; or
a pharmaceutically acceptable salt or prodrug of the compound;
wherein the compound of formula I is administered in an amount of from about 10 mg/m² per day to about 500 mg/m² per day for up to about 14 days starting on the first day of a 28 day cycle, and
(ii) a second component consisting of an injection solution comprising as an active ingredient a chemotherapeutic agent which is administered in amount of from about 10 mg/m² to about 500 mg/m² on the first day of a 28 day cycle, the 28 day cycle being repeated as long as the tumor remains under control.

44. The method of any one of claims 39-41 and 43, wherein the cancer is selected from the group consisting of breast cancer, ovarian cancer, colon cancer, pancreatic cancer, prostate cancer, gastric cancer, lymphoma, leukemia, skin carcinoma, lung carcinoma, head and neck carcinoma.

45. The method of claim 44, wherein the pharmaceutical composition is administered by the means of injection or intravenous infusion.
